(19) United States
(12) Patent Application Publication
(10) Pub. No.: US 2012/0141986 A1
(43) Pub. Date: Jun. 7, 2012

(54) Multivalent substrate elements for detection of nucleic acid sequences

(76) Inventors: Kenneth Kuhn, San Diego, CA (US); Timothy K. McDaniel, San Diego, CA (US)

(21) Appl. No.: 11/729,015

(22) Filed: Mar. 27, 2007

(51) Int. Cl. C12Q 1/68

(52) U.S. Cl. 435/6.11

(57) ABSTRACT

The invention provides a method of detecting multiple nucleic acid sequences using multiplex substrate elements, each having predetermined sets of independent probes, and using mistures of distinguishably labeled nucleotides.

### BACKGROUND OF THE INVENTION

This invention relates generally to methods for detecting nucleic acids and, more specifically to multiplex detection formats amenable to high throughput nucleic acid analysis.

The diagnosis and treatment of human diseases continues to be a major area of social concern. Improvements in health care are closely associated with a greater understanding of disease causes as well as improvements in the diagnosis and treatment of such diseases. Advancements from research and development have improved both the quality of life and life span of affected individuals. However significant, the progression of advancements from research and development has been slow and painstaking.

Further complications in the progression of scientific advancements and its practical medical application can result from technical limitations in available methodology. Many times, continued progress can be stalled due to the unavailability or insufficiency in technological sophistication needed to continue studies or implement practical applications at the new extremes. Therefore, further advancements from scientific discoveries to the medical field necessarily have to await progress in other fields for the advent of more capable technologies and materials. As a result, advancements having practical diagnostic and therapeutic applications can occur relatively slowly.

Genomic technology has been one such scientific advancement purported to open new avenues into the medical diagnostic and therapeutic fields. Genomic research has resulted in the sequencing of numerous whole genomes, including human, and has spurred futuristic speculation for diagnostic medical applications because of the availability of complete genome sequences. However, the application of the vast amount of genomic information and technology to medical diagnosis and treatment appears to still be in its infancy. One drawback hindering the application of genomics to practical medicine is the inability to efficiently generate and process large amounts of accurate sequence information amenable to diagnostic settings.

Thus, there exists a need for a nucleic acid detection process amenable to clinical settings that increases the efficiency and accuracy of high throughput analysis. The present invention satisfies this need and provides related advantages as well.

### SUMMARY OF THE INVENTION

The invention provides a multiplex substrate element, including an attached first nucleic acid and an attached second nucleic acid, the first nucleic acid including a first target specific probe, a hybridized first target nucleic acid and a first nucleotide having a first label indicative of the first target nucleic acid, the attached second nucleic acid including a second target specific probe, a hybridized second target nucleic acid and a second nucleotide having a second label indicative of the second target nucleic acid, wherein the first target nucleic acid has a sequence that is different from the second target nucleic acid, and wherein the first label is distinctive from the second label.

The invention also provides a population of modified target specific probes including a plurality of different multiplex substrate elements, each element including an attached first nucleic acid and an attached second nucleic acid, the first nucleic acid includes a first target specific probe, a hybridized first target nucleic acid and a first nucleotide having a first label indicative of the first target nucleic acid, the attached second nucleic acid including a second target specific probe, a hybridized second target nucleic acid and a second nucleotide having a second label indicative of the second target nucleic acid, wherein the first target nucleic acid has a sequence that is different from the second target nucleic acid, and wherein the first label is distinctive from the second label. The population can further include a multiplex substrate element including an attached third nucleic acid including a third target specific probe, a hybridized third target nucleic acid and a third nucleotide having a third label indicative of the third target nucleic acid, and an attached fourth nucleic acid including a fourth target specific piobe, a hybridized fourth target nucleic acid and a fourth nucleotide having a fourth label indicative of the fourth target nucleic acid, wherein the third target nucleic acid has a sequence that is different from the first, second and fourth target nucleic acids, wherein the fourth target nucleic acid has a sequence that is different from the first, second and third target nucleic acids, and wherein the third label is distinctive from the fourth label.

Further provided is method of detecting nucleic acid sequences. The method can include the steps of (a) contacting under conditions sufficient for hybridization a population of target nucleic acids with a plurality of multiplex substrate elements, each element including an attached first nucleic acid and an attached second nucleic acid, the first nucleic acid including a first target specific probe, the second nucleic acid including a second target specific probe, thereby forming hybridization complexes including the first target specific probe with a first target nucleic acid and the second target specific probe with a second target nucleic acid, wherein the first target nucleic acid has a sequence that is different from the second target nucleic acid; (b) contacting the hybridization complexes with a polymerase and a nucleotide mixture to modify at least one of the target specific probes, thereby forming at least one modified target specific probe, the nucleotide mixture containing at least two nucleotides having first and second distinct labels, respectively, and (c) determining incorporation of the first or second label into the at least one modified target specific probe, thereby determining the presence or absence of the first or second target sequences.

The invention provides a method of detecting nucleic acid sequences. The method can include the steps of (a) contacting under conditions sufficient for hybridization a population of target nucleic acids with a plurality of multiplex substrate elements including at least first and second multiplex substrate elements; (i) the first element including an attached first nucleic acid and an attached second nucleic acid, the first nucleic acid including a first target specific probe and the second nucleic acid including a second target specific probe; (ii) the second element including an attached third nucleic acid and an attached fourth nucleic acid, the third nucleic acid including a third target specific probe and the fourth nucleic acid including a fourth target specific probe, thereby forming hybridization complexes including the first target nucleic acid and the first target specific probe, the second target nucleic acid and the second target specific probe, the third target nucleic acid and the third target specific probe and the fourth target nucleic acid and the fourth target specific probe; (b) contacting the hybridization complexes with a polymerase and a nucleotide mixture to modify at least one of the target specific probes attached to the first multiplex substrate element and to modify at least one of the target specific probes attached to the second multiplex substrate element, thereby forming at least two modified target specific probes, the nucleotide mixture containing at least two nucleotides having first and second distinct labels, respectively, and (c) determining incorporation of the first or second labels into the modified target specific probes, thereby determining the presence or absence of the first, second, third or fourth target sequences.

A kit is provided. The kit can include (a) a plurality of multiplex substrate elements, each of the multiplex substrate elements including an attached first nucleic acid and an attached second nucleic acid, the first nucleic acid including a first target specific probe and a second nucleic acid including a second target specific probe, and (b) two or more different nucleotides having distinct labels.

Also provided is a method of evaluating quality of an array of multiplex substrate elements. The method can include the steps of (a) providing an array including a population of multiplex substrate elements including at least a first and a second subpopulation, wherein the multiplex substrate elements of each subpopulation include: (i) first nucleic acid including a first target specific probe and a first identifier sequence, and (ii) second nucleic acid including a second target specific probe and a second identifier sequence, wherein the first and second nucleic acids are attached to the same multiplex substrate elements; (b) detecting both the first and second identifier sequences to decode the position of each of the target specific probes on the array, and (c) determining whether the amount of each hybridizable target specific probe at each multiplex substrate element is sufficient to pass a quality metric, wherein the amount of each the first and second identifier sequence at each multiplex substrate element correlates with the amount of each target specific probe available for hybridization at each multiplex substrate element.

A method is provided for identifying a plurality of target nucleic acid sequences. The method can include the steps of (a) obtaining signals from a plurality of multiplex substrate elements, each of the multiplex substrate elements including two different target specific probes, the signals including a first signal indicative of a first type of nucleotide in a first target nucleic acid and a second signal indicative of a second type of nucleotide in a second target nucleic acid, wherein the signals are distinguishable from each other, and wherein the first type of nucleotide is different from the second type of nucleotide; (b) providing nucleotide sequences for the two different target specific probes at each of the multiplex substrate elements; (c) determining the presence or absence of the first signal and the second signal at each of the multiplex substrate elements, wherein at least a subset of the multiplex substrate elements produce the first signal and the second signal, thereby determining the type of nucleotide at each of the multiplex substrate elements, and (d) correlating the nucleotide sequences for the two different target specific probes with the type of nucleotide at each of the multiplex substrate elements, thereby identifying the nucleotide sequences of the first target nucleic acid sequence and the second target nucleic target sequence at each of the multiplex elements.

### BRIEF DESCRIPTION OF THE DRAWINGS

FIG. 1 shows a nucleic acid detection assay scoring single nucleotide polymorphisms (SNP) that employs four different labels where each multiplex substrate element contains different attached probes.

FIG. 2 shows a nucleic acid detection assay scoring SNPs that employs two different labels where each multiplex substrate element contains different attached probes.

FIG. 3 shows a bipartite identifier sequence attached to a multiplex substrate element of the invention.

### DETAILED DESCRIPTION OF THE INVENTION

This invention is directed to compositions and methods for increasing the multiplex capability of substrate elements within a microarray. Increased multiplex capability reduces the number of required substrate elements for a particular determination and allows a greater number of measurements to be made per assay or per input substrate element. The invention is particularly useful in nucleic acid diagnostic settings because it combines label management with reduced usage of microarray elements, which allows for efficient simultaneous detection of large pluralities of target sequences. The invention also is useful in a wide range of different types of detection assays and with a wide range of target sequence numbers because the compositions and methods are scaleable. The number of substrate elements can be scaled up to accommodate greater numbers of target sequences or equally scaled down to accommodate small numbers of target sequences or single determinations. The number of target specific probes attached to a multiplex substrate element of the invention also can be scaled upwards to include greater than two different probes attached to the same multiplex substrate element. Scalability in either or both modes is particularly useful because it allows for flexible, efficient and accurate multiplex determination employing a wide variety of nucleic acid detection assays. Therefore, the compositions and methods of the invention can be tailored to suit a wide variety of detection needs.

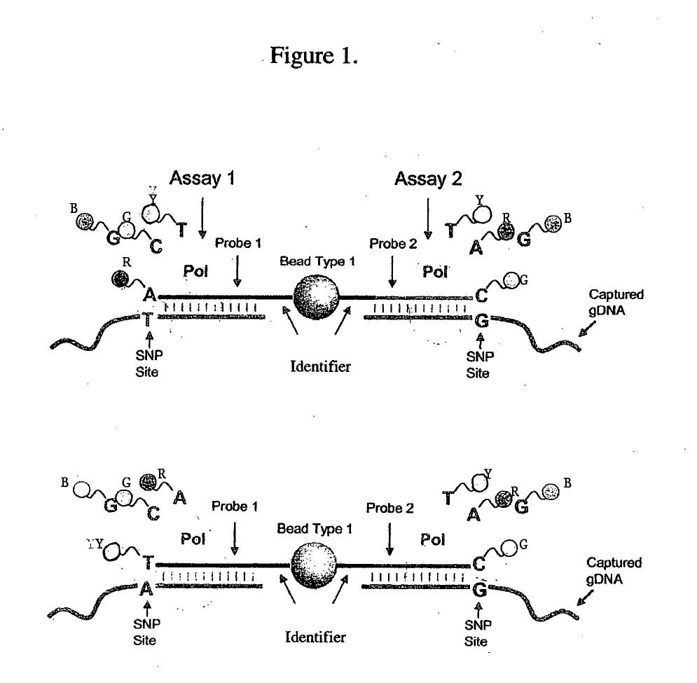

In one embodiment, the invention employs a pair of multiplex substrate elements, each element having two different target specific probes, and a label management system employing target-specific detection of four possible variants using four distinct labels. Nucleic acid detection occurs through scoring of label incorporation into a single target specific probe. In the specific example of single nucleotide polymorphism (SNP) detection, different alleles for two separate biallelic SNP loci can be distinguished using a single substrate element and four separate labels. As shown in FIG. 1, a substrate element can have probes to two different loci (i.e. probe 1 is directed to a first locus and probe 2 is directed to a second locus). The identity of the incorporated label determines the allele at each SNP locus. Hence, a single target specific probe hybridizes to all possible alleles at a locus and the SNP allele present in the target is determined based on which of four labels is incorporated at the probe.

In the above specific embodiment, the four labels can be managed such that nucleotides adenine (A), cytosine (C), guanine (G) and thymidine (T) (or analogs thereof such as uracil (U) which can be used in place of T) each have a distinct label. Taking the configuration of FIG. 1 as an example, a sample that is homozygous for the T allele at an [A/T] SNP targeted by probe 1 would produce signal at bead type 1 due to incorporation of the labeled A nucleotide. However, if the sample were heterozygous, having both A and T alleles present, then bead type 1 would produce two different signals due to incorporation of the labeled A nucleotide and labeled T nucleotide. For simplicity of explanation FIG. 1 illustrates the heterozygous case using separate pictures of the bead; however, typically the bead would have multiple copies of probe 1 and both labeled nucleotides would co-localize to the same bead. Two different loci can be detected at each substrate element because the probes and labels are managed such that the class of biallelic SNP that is targeted by the first probe on the element is different from the class of biallelic SNP targeted by the second probe on the element (i.e. probe 1 is specific for a locus having an [A/T] SNP class and probe 2 is specific for a locus having a [G/C] SNP class). Application of this specific embodiment to SNP detection allows any or all of the four nucleotide sequences possible at the SNP to be determined in a single measurement. Inclusion of multiple, different target specific probes on a single multiplex substrate further allows simultaneous detection of two or more different sequences in a single determination. Scaling of this multiplex capability can be implemented to simultaneously measure a very large population of target nucleic acids in a single assay.

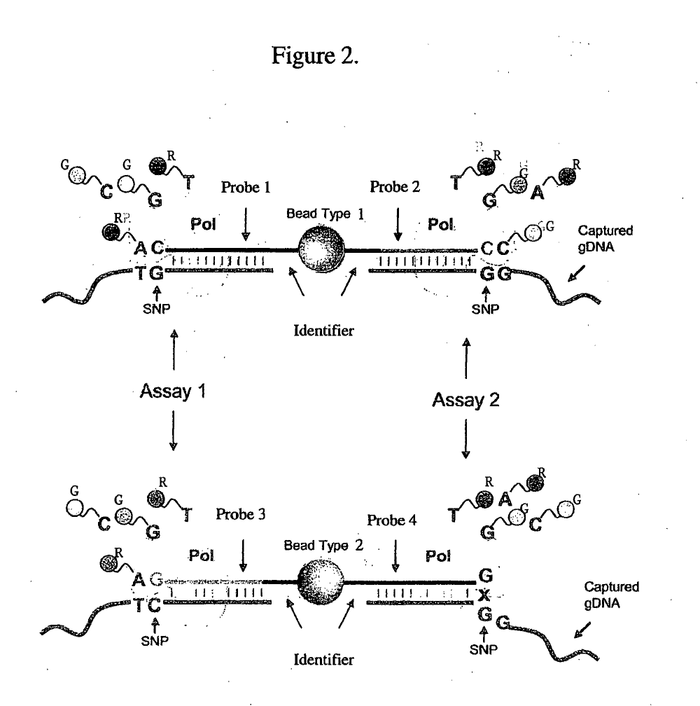

In a further embodiment, the invention employs a multiplex substrate element having two different target specific probes and a label management system employing target-specific detection of four possible variants using two distinct labels. Nucleic acid detection occurs through the scoring of label incorporation into either or both of the target specific probes. In the specific example of single nucleotide polymorphism (SNP) detection, different alleles for two separate biallelic SNP loci can be distinguished using only two different substrate elements and as few as two different labels. As shown in FIG. 2, the two substrate elements can be configured such that each element has probes to two different loci and to only one allele of each of those loci (i.e. probe 1 is directed to the G allele of a first locus and probe 2 is directed to the G allele of a second locus). For each locus, the pair of probes used to distinguish different alleles are present on different elements (i.e. in FIG. 2, probe 1 and probe 3 are directed to the G and C alleles, respectively, of the same locus). Identification of which allele is present for a particular locus is determined according to presence or absence of signal at one or both elements. As shown in FIG. 2, a sample that is [G/C] heterozygous at the locus targeted by probes 1 and 3 would produce signal at both bead type 1 and bead type 2 (due to incorporation of label at probe 1 and at probe 3). However, if the sample had been homozygous at this locus then signal would only be produced from one of the bead types (i.e. if the sample were homozygous for the G allele then bead type 1 would produce signal due to incorporation of the label on probe 1 and no signal would be produced from bead type 2 since probe 3 is not labeled). Two different loci can be detected at each substrate element because the labels are managed such that the two probes that are on the same element are associated with a different label in the presence of their respective alleles (i.e. the label added to probe 1 is spectroscopically distinguishable from the label added to probe 2).

As used herein, the term “multiplex substrate element” is intended to mean a particle or region of a support that isolates together two or more different analytes within a population of different analytes contained in a common chamber. Isolation allows for simultaneous analysis of the two or more different analytes within the population. The population can be random or ordered. Exemplary multiplex substrate elements include microspheres and array or microarray features, such as spots contained on a slide, chip or other planar substrate. A multiplex substrate element also includes a particle or support that isolates together two or more different macromolecules or other polymers within a population of macromolecules or polymers contained in a common chamber. Therefore, a multiplex substrate element can be used for analytes such as nucleic acids, polypeptides, carbohydrates or for a wide variety of chemical analytes or polymers.

As used herein, the term “solid support” is intended to mean a substrate. The term includes any material that can serve as a solid or semi-solid foundation for attachment of probes, other nucleic acids and/or other polymers, including biopolymers. A solid support of the invention is modified, for example, or can be modified to accommodate attachment of probes or nucleic acids by a variety of methods well known to those skilled in the art. Exemplary types of materials including solid supports include glass, modified glass, functionalized glass, inorganic glasses, microspheres, including inert and/or magnetic particles, plastics, polysaccharides, nylon, nitrocellulose, ceramics, resins, silica, silica-based materials, carbon, metals, an optical fiber or optical fiber bundles, a variety of polymers other than those exemplified above and multiwell microtiter plates. Specific types of exemplary plastics include acrylics, polystyrene, copolymers of styrene and other materials, polypropylene, polyethylene, polybutylene, polyurethanes and Teflon™. Specific types of exemplary silica-based materials include silicon and various forms of modified silicon.

The term “microsphere,” “bead” or “particle” refers to a small discrete solid support of the invention. Populations of discrete solid supports can be used for attachment of populations of probes or other nucleic acids such that individual supports in the population differ from each other with regard to the species of probe(s) that is attached. The composition of a microsphere can vary, depending on, for example, the format, chemistry and/or method of attachment and/or on the method of nucleic acid synthesis. Exemplary microsphere compositions include solid supports, and chemical functionalities imparted thereto, used in polynucleotide, polypeptide and/or organic moiety synthesis. Such compositions include, for example, plastics, ceramics, glass, polystyrene, methylstyrene, acrylic polymers, paramagnetic materials, thoria sol, carbon graphite, titanium dioxide, latex or cross-linked dextrans such as Sepharose, cellulose, nylon, cross-linked micelles and Teflon™, as well as any other materials that can be found described in, for example, “Microsphere Detection Guide” from Bangs Laboratories, Fishers Ind.

The geometry of a microsphere also can correspond to a wide variety of different forms and shapes. For example, microspheres used as solid supports of the invention can be spherical, cylindrical or can have any other geometrical shape and/or irregular shape. In addition, microspheres can be, for example, porous, thus increasing the surface area of the microsphere available for probe or other nucleic acid attachment. Exemplary sizes for microspheres used as solid supports in the methods and compositions of the invention can range from nanometers to millimeters or from about 10 nm to 1 mm. Particularly useful sizes include microspheres from about 0.2 μm to about 200 μm and from about 0.5 μm to about 5 μm being particularly useful.

In particular embodiments, microspheres or beads can be arrayed or otherwise spatially distinguished. Exemplary bead-based arrays that can be used in the invention include, without limitation, those in which beads are associated with a solid support such as those described in U.S. Pat. No. 6,355,431 B1, US 2002/0102578 and PCT Publication No. WO 00/63437. Beads can be located at discrete locations, such as wells, on a solid-phase support, whereby each location accommodates a single bead. Alternatively, discrete locations where beads reside can each include a plurality of beads as described in, for example, U.S. patent application Nos. US 2004/0263923, US 2004/0233485, US 2004/0132205 or US 2004/0125424. Beads can be associated with discrete locations via covalent bonds or other non-covalent interactions such as gravity, magnetism, ionic forces, van der Waals forces, hydrophobicity or hydrophilicity. However, the sites of an array of the invention need not be discrete sites. For example, it is possible to use a uniform surface of adhesive or chemical functionalities that allows the attachment of particles at any position. Thus, the surface of an array substrate can be modified to allow attachment or association of microspheres at individual sites, whether or not those sites are contiguous or non-contiguous with other sites. Thus, the surface of a substrate can be modified to form discrete sites such that only a single bead is associated with the site or, alternatively, the surface can be modified such that a plurality of beads populates each site.

Beads or other particles can be loaded onto array supports using methods known in the art such as those described, for example, in U.S. Pat. No. 6,355,431. In some embodiments, for example when chemical attachment is done, particles can be attached to a support in a non-random or ordered process. For example, using photoactivatible attachment linkers or photoactivatible adhesives or masks, selected sites on an array support can be sequentially activated for attachment, such that defined populations of particles are laid down at defined positions when exposed to the activated array substrate. Alternatively, particles can be randomly deposited on a substrate. In embodiments where the placement of probes is random, a coding or decoding system can be used to localize and/or identify the probes at each location in the array. This can be done in any of a variety of ways, for example, as described in U.S. Pat. No. 6,355,431 or WO 03/002979. A further encoding system that is useful in the invention is the use of diffraction gratings as described, for example, in US Pat. App. Nos. US 2004/0263923, US 2004/0233485, US 2004/0132205, or US 2004/0125424.

An array of beads useful in the invention can also be in a fluid format such as a fluid stream of a flow cytometer or similar device. Exemplary formats that can be used in the invention to distinguish beads in a fluid sample using microfluidic devices are described, for example, in U.S. Pat. No. 6,524,793. Commercially available fluid formats for distinguishing beads include, for example, those used in XMAP™ technologies from Luminex or MPSS™ methods from Lynx Therapeutics.

Any of a variety of arrays known in the art can be used in the present invention. For example, arrays that are useful in the invention can be non-bead-based. A useful array is an Affymetrix™ GeneChip™ array. GeneChip™ arrays can be synthesized in accordance with techniques sometimes referred to as VLSIPS™ (Very Large Scale Immobilized Polymer Synthesis) technologies. Some aspects of VLSIPS™ and other microarray and polymer (including polypeptide) array manufacturing methods and techniques have been described in U.S. Pat. No. 09/536,841, International Publication No. WO 00/58516; U.S. Pat. Nos. 5,143,854, 5,242,974, 5,252,743, 5,324,633, 5,445,934, 5,744,305, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846, 6,022,963, 6,083,697, 6,291,183, 6,309,831 and 6,428,752; and in PCT Applications Nos. PCT/US99/00730 (International Publication No. WO 99/36760) and PCT/US01/04285. Such arrays can hold over 500,000 probe locations, or features, within a mere 1.28 square centimeters. The resulting probes are typically 25 nucleotides in length.

A spotted array also can be used in a method of the invention. An exemplary spotted array is a CodeLink™ Array previously available from Amersham Biosciences. CodeLink™ Activated Slides are coated with a long-chain, hydrophilic polymer containing amine-reactive groups. This polymer is covalently crosslinked to itself and to the surface of the slide. Probe or other nucleic acid attachment can be accomplished through covalent interaction between the amine-modified 5′ end of the oligonucleotide probe and the amine reactive groups present in the polymer. Probes or other nucleic acids can be attached at discrete locations (i.e. features or substrate elements) using spotting pens. Such pens can be used to create features having a spot diameter of, for example, about 140-160 microns. In a specific embodiment, nucleic acid probes at each spotted feature can be 30 nucleotides long.

Another array that is useful in the invention is one manufactured using inkjet printing methods such as SurePrint™ Technology available from Agilent Technologies. Such methods can be used to synthesize probes or other nucleic acids in situ or to attach presynthesized nucleic acids having moieties that are reactive with a substrate surface. A printed microarray can contain about 22,575 features on a surface having standard slide dimensions (about 1 inch by 3 inches). Generally, the printed nucleic acids are 25 or 60 nucleotides in length. Also useful are arrays manufactured by Nimblegen (Reykjavik, Iceland) or by Xeotron methods (available from Invitrogen, Carlsbad, Calif.).

It will be understood that the specific synthetic methods and probe or other nucleic acid lengths described above for different commercially available arrays are merely exemplary. Similar arrays can be made using modifications of the methods and nucleic acids having other lengths such as those set forth herein can also be placed at each feature of the array.

Those skilled in the art will know or understand that the composition and geometry of a solid support of the invention can vary depending on the intended use and preferences of the user. Therefore, although microspheres and chips are exemplified herein for illustration, given the teachings and guidance provided herein, those skilled in the art will understand that a wide variety of other solid supports exemplified herein or well known in the art also can be used in the methods and/or compositions of the invention.

Target specific probes or identifier sequences, for example, can be attached to a solid support of the invention using any of a variety of methods well known in the art. Such methods include for example, attachment by direct chemical synthesis onto the solid support, chemical attachment, photochemical attachment, thermal attachment, enzymatic attachment and/or absorption. These and other methods are will known in the art and are applicable for attachment of target specific probes or identifier sequences in any of a variety of formats and configurations. The resulting target specific probes or identifier sequences can be attached to a solid support via a covalent linkage or via non-covalent interactions. Exemplary non-covalent interactions are those between a ligand-receptor pair such as streptavidin (or analogs thereof) and biotin (or analogs thereof) or between an antibody and epitope. Once attached to the first solid support, the target specific probes are amenable for use in the methods and compositions as described herein.

As used herein, the term “target specific probe” is intended to mean a molecule having sufficient affinity to specifically bind to a target molecule. An exemplary target specific probe is a polynucleotide having sufficient complementarity to specifically hybridize to a target nucleic acid. A target specific probe functions as an affinity binding molecule for isolation or analysis of a target molecule (such as a nucleic acid) from other molecules in a population. Target specific probes of the invention are attached, or can be modified to attach, to a solid support. The attachment can be directly to the solid support or indirectly such as through one or more identifier sequences. Target specific probes can be of any desired length and/or sequence so long as they exhibit sufficient complementarity to specifically hybridize to a target nucleic acid for isolation, including analysis or nucleotide sequence detection. Methods and target specific probe components for a variety of nucleic acid analysis and/or detection formats are well known to those skilled in the art.

A target specific probe or other nucleic acid used in a method of the invention can have any of a variety of compositions or sizes, so long as it has the ability to hybridize to a target nucleic acid with sequence specificity. Accordingly, a nucleic acid having a native structure or an analog thereof can be used. A nucleic acid with a native structure generally has a backbone containing phosphodiester bonds and can be, for example, deoxyribonucleic acid or ribonucleic acid. An analog structure can have an alternate backbone including, without limitation, phosphoramide, phosphorothioate, phosphorodithioate, O-methylphophoroamidite linkages, and peptide nucleic acid backbones and. Other analog structures include those with positive backbones (see, for example, Dempcy et al., Proc. Natl. Acad. Sci. USA 92:6097 (1995); non-ionic backbones (see, for example, U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994) and non-ribose backbones, including, for example, those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Analog structures containing one or more carbocyclic sugars are also useful in the methods and are described, for example, in Jenkins et al., Chem. Soc. Rev. (1995) pp169-176. Several other analog structures that are useful in the invention are described in Rawls, C & E News Jun. 2, 1997 page 35. Locked nucleic acids can also be used.

As used herein, the term “population,” when used in reference to nucleic acids is intended to mean two or more different nucleic acids having different nucleotide sequences. When used in reference to a multiplex substrate element, the term is intended to mean two or more different elements containing a different plurality of attached nucleic acids. Therefore, a population constitutes a plurality of two or more different members. Populations can range in size from small, medium, large, to very large. The size of small populations can range, for example, from a few members to tens of members. Medium populations can range, for example, from tens of members to about 100 members or hundreds of members. Large populations can range, for example, from about hundreds of members to about 1000 members, to thousands of members and up to tens of thousands of members. Very large populations can range, for example, from tens of thousands of members to about hundreds of thousands, a million, millions, tens of millions and up to or greater than hundreds of millions members. Therefore, a population can range in size from two to well over one hundred million members as well as all sizes, as measured by the number of members, in between and greater than the above exemplary ranges. Specific examples of large populations include a plurality of target specific probes of about 5×10⁵ or 1×10⁶. Accordingly, the definition of the term is intended to include all integer values greater than two. An upper limit of a population of the invention can be set, for example, by the theoretical diversity of nucleotide sequences in a complex mixture of the invention. The term “each,” when used in reference to individuals within a population, is intended to recognize one or more individuals in a population. Unless explicitly stated otherwise the term “each” when used in this context is not necessarily intended to recognize all of the individuals in a population. Thus, “each” is intended to be an open term.

As used herein, the term “identifier sequence” is intended to mean a unique sequence associated with a target specific probe or other nucleic acid. An identifier sequence functions as a unique tag which is used to identify the associated target specific probe by inseparable correlation. The term is intended to include combinations of unique sequences that can be concatenated to form, for example, bipartite, tripartite or other multipartite sequence structures. The different portions of such multipartite identifier sequences can be joined together or physically separated on, for example, a solid support or other multiplex substrate element of the invention. An identifier sequence will have a nucleotide sequence, or a portion of a nucleotide sequence, that is different or distinguishable from the nucleotide sequence of its associated target specific probe. The sequence can be synthetic or naturally occurring and the lengths and/or nucleotide characteristics will include any of those described herein for other nucleic acids of the invention. For example, an identifier sequence can have sizes ranging between, for example, 10-100 nucleotides (nt) or more, or have a native phosphodiester backbone, an analog structure or a combination thereof. Given the teachings and guidance provided herein, those skilled in the art will know that a wide variety of designs and nucleotide sequences can be used to generate a diversity of nucleic acids which can be employed as unique tags for target specific probes.

As used herein, the term “target nucleic acid” is intended to mean a nucleic acid analyte. Particular forms of nucleic acid analytes of the invention include any type of nucleic acids found in an organism. For example, a target nucleic acid that is applicable for analysis using the methods and compositions of the invention include genomic DNA (gDNA), expressed sequence tags (ESTs), DNA copied messenger RNA (cDNA), RNA copied messenger RNA (cRNA), mitochondrial DNA or genome, RNA, messenger RNA (mRNA) and/or other populations of RNA. Furthermore, nucleic acid products of amplification reactions using any of the foregoing nucleic acid species can be used as a target nucleic acid. For example, a target nucleic acid used in a method of the invention can be an amplicon produced from DNA such as gDNA or cDNA, or an amplicon produced from RNA such as mRNA or cRNA. Fragments and/or portions of these exemplary target nucleic acids also are included within the meaning of the term as it is used herein.

It will be understood that a locus or allele of a nucleic acid can be evaluated in a method of the invention using probes that hybridize to the nucleic acid, its complement or an amplicon of the nucleic acid. Identification of the nucleotide composition or sequence of an allele in a nucleic acid will typically be understood to identify the composition or sequence for the nucleic acid, its complement, a template from which it was amplified and an amplicon produced from either or both strands of the nucleic acid.

The compositions and methods set forth herein are useful for analysis of large genome nucleic acid analytes such as those typically found in eukaryotic unicellular and multicellular organisms. Exemplary eukaryotic target nucleic acids that can be used in a method of the invention includes, without limitation, that from a mammal such as a rodent, mouse, rat, rabbit, guinea pig, ungulate, horse, sheep, pig, goat, cow, cat, dog, primate, human or non-human primate; a plant such as Arabidopsis thaliana, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a dictyostelium discoideum; a fungi such as pneumocystis carinii, Takifugu rubripes, yeast, Saccharamoyces cerevisiae or Schizosaccharomyces pombe; or a plasmodium falciparum. The compositions and methods of the invention also can be used with target nucleic acids from organisms having smaller genomes such as those from a prokaryote such as a bacterium, Escherichia coli, staphylococci or mycoplasma pneumoniae; an archae; a virus such as Hepatitis C virus or human immunodeficiency virus; or a viroid.

A target nucleic acid can be isolated from one or more cells, bodily fluids or tissues. Known methods can be used to obtain a bodily fluid such as blood, sweat, tears, lymph, urine, saliva, semen, cerebrospinal fluid, feces or amniotic fluid. Similarly known biopsy methods can be used to obtain cells or tissues such as buccal swab, mouthwash, surgical removal, biopsy aspiration or the like. Target nucleic acids also can be obtained from one or more cell or tissue in primary culture, in a propagated cell line, a fixed archival sample, forensic sample, fresh frozen paraffin embedded sample or archeological sample.

Exemplary cell types from which target nucleic acids can be obtained include, without limitation, a blood cell such as a B lymphocyte, T lymphocyte, leukocyte, erythrocyte, macrophage, or neutrophil; a muscle cell such as a skeletal cell, smooth muscle cell or cardiac muscle cell; germ cell such as a sperm or egg; epithelial cell; connective tissue cell such as an adipocyte, fibroblast or osteoblast; neuron; astrocyte; stromal cell; kidney cell; pancreatic cell; liver cell; or keratinocyte. A cell from which gDNA is obtained can be at a particular developmental level including, for example, a hematopoietic stem cell or a cell that arises from a hematopoietic stem cell such as a red blood cell, B lymphocyte, T lymphocyte, natural killer cell, neutrophil, basophil, eosinophil, monocyte, macrophage, or platelet. Other cells include a bone marrow stromal cell (mesenchymal stem cell) or a cell that develops therefrom such as a bone cell (osteocyte), cartilage cells (chondrocyte), fat cell (adipocyte), or other kinds of connective tissue cells such as one found in tendons; neural stem cell or a cell it gives rise to including, for example, a nerve cells (neuron), astrocyte or oligodendrocyte; epithelial stem cell or a cell that arises from an epithelial stem cell such as an absorptive cell, goblet cell, Paneth cell, or enteroendocrine cell; skin stem cell; epidermal stem cell; or follicular stem cell. Generally any type of stem cell can be used including, without limitation, an embryonic stem cell, adult stem cell, or pluripotent stem cell.

The invention provides a multiplex substrate element having a solid support containing a first nucleic acid including an identifier sequence and a first target specific probe and a second nucleic acid including an identifier sequence and a second target specific probe. The solid support can include, for example a microsphere.

The compositions and methods of the invention can employ a multiplex substrate element where, for example, target specific probes can be attached in a variety of configurations. Multiplex embodiments of the invention employ attachment of two or more different target specific probes to a substrate element. The substrate element serves as a solid support that can be used in nucleic acid detection methods alone or as one element within a compilation or array of many different elements of a larger multiplex scheme. Each element within such a larger multiplex scheme serves as an individual detectable unit. Probes attached to an individual unit are typically not spatially resolved but individual detectable units can be resolved from each other allowing the sequences attached to different units within the entire compilation to be distinguished in a single assay. The compositions and methods of the invention provide for a scalable number of nucleic acid detection measurements corresponding to the number of different target specific sequences on a substrate element combined with the number of unique substrate elements. This scalability is due, at least in part, to configuring the location of probes in an array and partitioning labels between different target nucleic acids in accordance with the methods set forth herein.

In specific embodiments of the invention, the arrangement of substrate elements within a multiplex scheme can be ordered or random. Similarly, the invention can accommodate a variety of different attachment configuration for a target specific probe such as those set forth previously herein with regard to different microarray formats. In general, target specific probes are associated directly or indirectly with one or more identifier sequences that uniquely correlate a probe with a substrate element. Inclusion of identifier sequences therefore provides a link between the substrate element, its location within an array and the target specific probes attached to the substrate element. Immobilization of a plurality of target specific probes to substrate elements through identifier sequences is particularly useful because it allows for proportionate increases in the level of multiplexing to be achieved by enhancing the information content within each substrate element.

Multiplex substrate elements of the invention include a wide variety of solid supports or physical features within a microarray. Multiplex substrate elements of the invention also include a wide variety of physical objects within, for example, a liquid array such as the flow chamber of a flow cytometer. In general, a multiplex substrate element of the invention will be a support allowing attachment of two or more target specific probes and includes, for example, a feature contained on or within a solid support having many such features or an individual solid support that forms an individual feature. An array of features includes, for example, a component of a support that physically or functionally separates one element from another. The component separates the two or more target specific probes attached at a first feature from two or more target specific probes attached at a second feature. Accordingly, a multiplex substrate element includes a solid support having separable structural features contained in or attached to a support as well as a solid support that is itself a separable structural feature.

Separable structural features on a multiplex substrate element include, for example, spots on an array, as exemplified previously, as well as various other structural features useful for nucleic acid attachment to a solid support or structural features well known to those skilled in the art. For example, any of the modifications for nucleic acid attachment to solid supports described above or below can be used to generate separable features on solid supports such as a microarray or chip and can be employed as a multiplex substrate element of the invention. Other separable structural features useful as a multiplex substrate element of the invention include, for example, a patterned substrate such as wells etched into a slide or chip. The pattern of the etchings and geometry of the wells can take on a variety of different shapes and sizes so long as such features physically or functionally isolate the two or more target specific probes attached to or contained therein. Particularly useful supports having such structural features are patterned substrates that can select the size of solid support particles such as microspheres. An exemplary patterned substrate having these characteristics is the etched substrate used in connection with BeadArray technology (Illumina, Inc., San Diego, Calif.).

Solid supports useful as a multiplex substrate element apart from or together with a structural feature contained in or attached to a support include for example, particles, microspheres, beads and the like. In this specific embodiment, any substrate that can be used to attach two or more different target specific probes can be employed as a solid support in the multiplex compositions and methods of the invention. A wide variety of solid supports have been exemplified previously. Any of such solid supports can be used in the compositions or methods of the invention alone or in combination with another type of solid support exemplified herein or well known to those skilled in the art. While the invention is exemplified below by reference to microspheres, beads or particles, given the teachings and guidance provided herein, those skilled in the art will understand that any of the solid supports exemplified previously or others well known in the art that can provide a platform for attachment of two or more different nucleic acids are equally applicable for use in the compositions or methods of the invention.

Also for ease of illustration, the invention is exemplified herein by reference to nucleic acids. Given the teachings and guidance provided herein, those skilled in the art will understand that the methods and compositions of the invention are equally applicable to complex mixtures of biopolymers other than nucleic acids. In particular, it will be understood by those skilled in the art that the compositions and methods of the invention can be routinely employed for the analysis and detection of biopolymers other than nucleic acids including, for example, polypeptides, polysaccharides and/or lipids. Similarly, those skilled in the art also will understand from the teachings and guidance provided herein that the compositions and methods of the invention also can be equally employed with analysis and detection of a wide variety of nucleic acid or biopolymer characteristics other than primary sequence. For example, assays for detection of methylation, phosphorylation or other biopolymer modifications and/or moieties can be determined by, for example, substitution of the nucleotide sequence determinations exemplified herein with an applicable assay for the modification of interest. Therefore, a wide variety of biopolymer methods well known in the art for analysis, detection and/or sequence determination are applicable for use with the compositions and methods of the invention. Such methods can be used in lieu of a method of characterization exemplified herein or together with a characterization method exemplified herein. For example, both nucleotide sequence and methylation content or location can be determined using the multiplex compositions and methods of the invention. Sequence and modification content can be determined simultaneous, in parallel, in series and/or consecutively, for example.

A multiplex substrate element of the invention includes a solid support containing at least a first and second nucleic acid. Numerical modifiers such as the terms first, second, third, and fourth when used in reference to, for example, nucleic acids, nucleotide sequences or multiplex substrate elements refer to different species thereof, unless explicitly stated to the contrary. For example, reference to a first and a second nucleic acid means two nucleic acids having different nucleotide sequences, in contrast to two copies of a nucleic acid having the same sequence. Similarly, reference to first, second, third and fourth nucleic acids means four different nucleic acids each having a different sequence. A first and second nucleotide sequence refers to two different sequences rather than two identical sequences whereas a first and second solid support or multiplex substrate element refers to two supports each containing different nucleic acids compared to the other.

A multiplex substrate element of the invention can include one or more identifier sequences. As described further below with reference to the methods of the invention, an identifier sequence can impart information content onto the multiplex substrate element to uniquely correlate one or more target specific probes to a solid support, and/or to identify the element's location within an array or other multiplex configuration. An identifier sequence is therefore any sequence, moiety, ligand or other molecular handle that can be attached to the substrate element to uniquely identify its co-localized target specific target specific probe and, if desired, its location among a plurality of multiplex substrate elements. Accordingly, an identifier can be, for example, a unique nucleotide sequence used in connection with nucleic acid target specific probes for detection of nucleic acid analytes, a unique polypeptide used in connection with polypeptide affinity probes, for example, for detection of polypeptide analytes and/or a chemical moiety or other ligand used in connection with other target specific probes, for example, for detection of other biopolymers. Because an identifier sequence functions as a unique tag for its associated target specific probe, the compositions and methods of the invention also can employ various combinations of different types of identifier sequences and target specific probes. For example, nucleic acid identifier sequences can be used to tag polypeptide target specific probes where the multiplex detection methods utilize, for example, affinity binding for polypeptide detection and hybridization for detection of identifier sequences. Given the teachings and guidance provided herein, those skilled in the art will understand that a wide variety of combinations and permutations between types of identifier sequences and types of target specific probes can be utilized to effectively achieve detection of target analytes and identification to a multiplex substrate element.

With respect to the nucleic acid detection methods exemplified herein, one specific embodiment employs nucleic acid identifier sequences used in conjunction with nucleic acid target specific probes. In this configuration, hybridization detection steps can be utilized for both target nucleic acid and identifier sequence detection and/or identification. For purposes of illustration, this specific embodiment will be exemplified below.

Nucleic acid identifier sequences can be of any desired length and/or sequence of nucleotides so long as they exhibit sufficient complementarity to specifically hybridize to a complementary sequence used for identification. In specific embodiments of the invention, the complementary sequences used for identification are referred to as decoder probes because they decipher the associated target specific probe sequence and/or its location in relation to its associated substrate element within a larger multiplex scheme such as an array. Nucleic acid identifier sequences and their corresponding complementary decoder sequences generally will be designed and made to exhibit similar or the same characteristics for a particular assay. Identifier sequences function as a tag for the target specific probe whereas decoder sequences are complementary to its cognate identifier sequence and function as a molecular handle to identify and/or characterize the tag. Given the teachings and guidance provided herein, those skilled in the art will understand that the exemplary descriptions herein with respect to identifier sequences are equally applicable to their corresponding complementary sequences. Methods for identifier sequence design, synthesis, modification and/or attachment to a substrate element for a variety of nucleic acid analysis and/or detection formats exemplified herein are well known to those skilled in the art as described, for example, in Gunderson et al., Genome Research, 14: 870-877 (2004); U.S. Pat. No. 7,033,754 and US 2003/0157504, each of which is incorporated herein by reference.

An identifier sequence or other nucleic acid sequence used in a method of the invention can have any of a variety of compositions or sizes, so long as it has the ability to hybridize to its complimentary decoder probe sequence with specificity. Accordingly, a nucleic acid having a native structure or an analog thereof can be used. As described previously with respect to target specific probes, nucleic acids with native structures generally have backbones containing phosphodiester bonds and can be, for example, deoxyribonucleic acid or ribonucleic acid. An analog structure can have an alternate backbone including, for example, phosphoramide, phosphorothioate, phosphorodithioate, O-methylphophoroamidite linkages, and peptide nucleic acid backbones and. Other analog structures such as those described previous with respect to target specific probes also can be used (see, for example, Dempcy et al., supra; U.S. Pat. Nos. 5,386,023, 5,637,684, 5,602,240, 5,216,141 and 4,469,863, supra; Kiedrowshi et al., supra; Letsinger et al., supra; Letsinger et al., supra; Chapters 2 and 3, ASC Symposium Series 580, supra; Mesmaeker et al., supra; Jeffs et al., supra; U.S. Pat. Nos. 5,235,033 and 5,034,506, and Chapters 6 and 7, ASC Symposium Series 580, supra; Jenkins et al., supra, and Rawls, supra).

Selection of an identifier sequence to employ in a composition or method of the invention can entail designing and/or screening for the identifier sequence to be unique to its associated target specific probe relative to other target specific probes attached to different substrate elements. The identifier sequence can additionally be designed and/or selected from a screen to be unique to its associated target specific .probe relative to different target specific probes attached to the same substrate element. These unique sequences are associated with their cognate target specific probes and used as affinity binders to bind or hybridize with their particular complementary sequences for detection and identification of their associated target specific probes within a multiplex analysis and/or detection scheme.

Similarly, a population of identifier sequences employed with a plurality of substrate elements or used in a multiplex detection method of the invention can be selected depending on the number of different target nucleic acids, level of multiplexing and type of analysis and/or determination to be performed so as to uniquely correlate with its cognate target nucleic acid probe and substrate element. For example, a population of unique nucleic acid sequences can be generated where each nucleic acid is about nine or more nucleotides (nt) in length. Therefore, unique sequences for each target specific probe within a large population can be generated using, for example identifier sequences having about nine or more nucleotides. The length of identifier sequence nucleic acids can be correspondingly shorter for smaller populations. Those skilled in the art will understand that identifier sequences longer than nine nucleotides can, for example, increase efficiency and hybridization specificity because partial cross-hybridization can be avoided by increasing stringency. Accordingly, identifier sequences can be generated longer or shorter than about nine nucleotides and can be used in the compositions and methods of the invention including, for example, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 ,51, 52, 53, 54, 55, 56, 57, 58, 59, 60, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 or more nucleotides in length. In one particularly useful embodiment of the invention, an identifier sequence is between about 26-32 nucleotides, typically between about 28-30 nucleotides, and more typically about 29 nucleotides. In other useful embodiments, the identifier sequence is bipartite where each subregion is between about 13-15 nucleotides.

Identifier sequences can be designed de novo or be modeled from known sequences employing nucleic acid sequence information available from a variety of sources. De novo design includes, for example, designing or selecting a nucleotide sequence without restriction to, or independent of, known nucleic acid sequence. It can be rational design of a desired sequence or randomly selected or generated. In exemplary embodiments of the invention, identifier sequences are rationally designed and correlated with one or more target specific probes to obtain a unique association between identifier and probe. Identifier sequences also can be produced by generating random sequences using, for example, algorithms well known in the art and correlated with one or more target specific probes. Association of the identifier and the target specific probe can occur, for example, by synthesizing both component as a single nucleic acid, separately followed by coupling or by any of a variety of other formats and procedures well known to those skilled in the art. Alternatively, identifier sequences can be obtained by, for example, random synthesis of sequences and can be sequenced prior to correlation and association with target specific probes. The design and use of molecular tags functioning as identifier sequences in array formats are well known to those skilled in the art and can be found described in, for example, U.S. Pat. Nos. 7,033,754; 6,355,432; WO 2005/003304, and in the patents and publications referenced previously with respect to solid supports, microspheres and array technologies.

Given the teachings and guidance provided herein, those skilled in the art will understand that a wide variety of approaches and procedures can be implemented to design and generate identifier sequences and populations of identifier sequences to obtain the requisite number of different identifier sequences for unique association with one or more target specific probes. In addition to the approaches exemplified above, known nucleic acids also can be obtained and correlated with one or more target specific probes so long as the sequences of such nucleic acids are distinct from target probe sequences used in a particular multiplex assay setting. The known nucleic acids can be used intact or portions thereof can be synthesized and associated with one or more target specific probes. Alternatively, identifier sequences can be derived from known sequences and chemically synthesized for use as an identifier sequence.

Nucleotide sequence information for known nucleic acids is available from a variety of well known sources. For example, including, for example, user derived, public or private databases, subscription sources and on-line public or private sources. These sources also can be used, for example, to obtain sequence information for generation of the target specific probes of the invention. Exemplary public databases for obtaining genomic and gene sequences include, for example, dbEST-human, UniGene-human, gb-new-EST, Genbank, Gb_pat, Gb_htgs, Refseq, Derwent Geneseq and Raw Reeds Databases. Access or subscription to these repositories can be found, for example, at the following URL addresses: dbEST-human, gb-new-EST, Genbank, Gb_pat, and Gb_htgs at URL:ftp.ncbi.nih.gov/genbank/; Unigene-human at URL:ftp.ncbi.nih.gov/repository/UniGene/; Refseq at URL:ftp.ncbi.nih.gov/refseq/; Derwent Geneseq at URL:wwvv.derwent.com/geneseq/ and Raw Reads Databases at URL:trace.ensembl.org/. The nucleic acid sequence information additionally can be generated by a user and used directly or stored, for example, in a local database. Various other sources Well known to those skilled in the art for nucleic acid sequence information also exist and can similarly be used for generating, for example, populations of target specific probes and identifier sequences.

In particular embodiments where a population of multiplex substrate elements are produced or used in a detection method of the invention, each substrate element and attached target specific probe combination will include, for example, a different identifier sequence. The teachings and guidance provided above and below with respect to design and/or selection, generation and association with a particular identifier sequence is applicable to the production of any size population of identifier sequences. Briefly, the population of identifier sequences is designed to uniquely correlate with one or more target specific probes attached to the same substrate element as the identifier sequence. In order to be unique as to an associated target specific probe, the identifier sequence should be unique compared to other relevant identifier sequences within the population or be distinguishable from other relevant identifier sequences by methods well known in the art. For example, if the population of identifier sequences is desired to uniquely tag all target specific probes to, for example, all alleles associated with a particular disease then a population of identifier sequences should include at least one unique identifier for each type of substrate element. Similarly, populations having different identifier sequences sufficient to uniquely tag some or all types of substrate elements used for the determination of alleles associated with two, three or four or more pathological conditions, or to uniquely tag some or all alleles for one or more pathological conditions for multiple different individuals should include a like number of different identifier sequences to uniquely tag at least each substrate element employed in such assays.

In addition to primary sequence for the specific nucleic acid identifier sequences exemplified herein, identifier sequences can take on a wide variety of structures and configurations. For example, as exemplified previously, identifier sequences can include two or more portions to form, for example, bipartite, tripartite or other multipartite sequence structures. The portions can be contiguous, non-contiguous, linear, branched and, if desired, circular. Other exemplary structures or modalities include, for example, repeating units and/or multiple copies of a sequence or unit. The different portions can be linked or joined within the same molecule, joined with a target specific probe and/or included as separate molecules either joined or not joined with a target specific probe. All combinations and permutations of these exemplary identifier sequence structures and configurations also can be used in a multiplex substrate element of the invention. Those skilled in the art will understand that the complexity of the identifier sequence structure can be modulated according to the information content need or preference to confer unique tags onto the target specific probes of the invention.

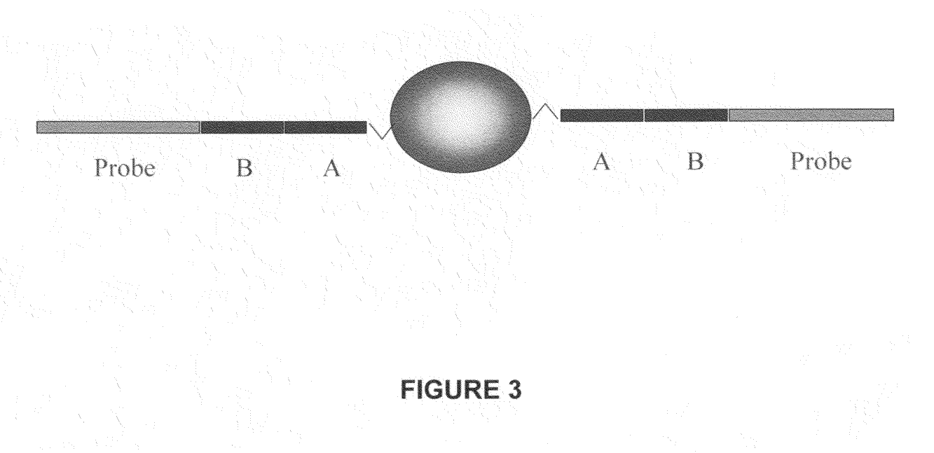

In one specific embodiment exemplifying multipartite identifier sequences, an identifier sequence contains two regions, referred to herein as A an B in FIG. 3. Both portions of this bipartite identifier sequence are attached to a single substrate element. For example, the first portion can include the A region sequence of the identifier and the second portion can include the B region sequence of that identifier. Identification of the substrate element, and its corresponding attached target specific probes, can then be ascertained using either the A region, the B region or both the A and B regions.

Multipartite identifier sequences are particularly useful in connection with random array formats because they can increase information content, allowing for a greater number of array features to be located for a given number of decoder labels (states) and decoding steps (stages) compared to the number of features that can be located when only a single identifier sequence is used as described, for example, in Gunderson et al., Genome Research, 14: 870-877 (2004); U.S. Pat. No. 7,033,754 and US 2003/0157504, each of which is incorporated herein by reference. In one exemplary embodiment, multiplex substrate elements are randomly ordered within an array and a hybridization-based identification or decoding scheme is used which employs predetermined combinations of two or more distinct subregions within an identifier sequence. Using this specific bipartite identifier sequence, each subregion attached to a substrate element can constitute a unique tag or combinations of subregions can be generated to create unique tags. For example, four unique subregions can be employed in pairs to generate two bipartite identifier sequences where each subregion constitutes a unique tag.

Deciphering bi- and other multi-partite identifier sequences to identify the target specific probe and/or its location within an array can employ any of the methods exemplified herein for decoding randomly ordered arrays. Such methods are exemplified below in reference to the methods of the invention. Other methods well known in the art also are equally applicable. In the multipartite identifier embodiments of the invention, decoding also can be usefully employed for confirming nucleic acid attachment to substrate elements. For example, employing a decoding scheme requiring both subregions of, for example, a bipartite identifier sequence for correct decoding of the element can be implemented for this purpose where the subregions are separately attached to the element. Detection of both subregions of the identifier sequence identifies both element type (i.e., which target specific probes are attached to the element) and also serves as an assurance that both immobilized subregions are present in adequate amounts to yield a robust hybridization signal. This internal control results because if one of the probes is not present on the substrate element then the element fails decoding and is ignored or discarded for subsequent detection steps.

Additionally, the relative amounts of each hybridizable target specific probe linked to each subregion on a particular element can be estimated or determined based on the signal arising from the complementary decoders that hybridize to each of the two identifier sequence subregions. If the relative amount of one probe to another is determined to be within an acceptable range based on comparison of the signals arising from their complementary decoders then the subregion can be designated as passing quality control. Alternatively, if the relative amount of one probe to another is outside of an acceptable range then the subregion can be considered to fail. Subregions that are passing can be subsequently used in analytical determinations whereas those that fail can be discarded or ignored during one or more subsequent analytical process. A substrate with an unacceptable number of failed subregions can be discarded or otherwise avoided in subsequent analytical methods. The range of acceptable differences between signals arising from a pair of decoders can be determined based on a number of factors such as the precision with which decoder signal correlates with the amount of their respective targets present at a substrate element. For example, if the base composition or melting temperature is substantially different between pairs of decoders being compared then the range of acceptable signal value differences can be wide compared to the range that is acceptable when the two decoders being compared are known to have similar behavior during hybridization and detection.

The multiplex substrate elements of the invention additionally include at least an attached first and second target specific probe. Each probe will be specific to the particular analytes of interest that are to be detected. Each target specific probe also will be designed or selected to be compatible with a particular detection format or multiplex configuration. Therefore, target specific probes can consist of a variety of different types of molecules as exemplified previously including, for example, polypeptide, affinity binding molecules and/or nucleic acid and the like. Target specific probes also can consist of a variety of different structures and formats depending on, for example, the detection method employed and the measurement objectives. For example target specific probes employing affinity binding molecules including antibodies, ligands and the like, can employ direct binding through the probe and the analyte. Alternatively, secondary binding formats can be employed where a primary probe having, for example, an affinity tag binds to the analyte and the probe attached to the substrate element binds to the affinity tag. A wide variety of primary and secondary probes as well as formats and configurations for such direct or indirect detection of an analyte are well known in the art and can be equally employed in the methods of the invention.

With reference to nucleic acids as an exemplary and illustrative embodiment, nucleic acid target probes specific to nucleic acid analytes similarly can take on a variety of structures, formats and configurations depending on the detection method and measurement objectives. In one specific embodiment where determination of the presence or absence of a nucleic acid analyte is desired, a target specific probe will be sufficient in length and complementarity to specifically hybridize to the target analyte. In another specific embodiment where single nucleotide changes in a target analyte are to be determined, such as for detection of single nucleotide polymorphisms, in addition to being sufficient in length and sequence complementarity, the probe also can be designed to contain a detection position for the SNP. As exemplified further below with reference to the methods of the invention, the location of the detection position can vary and the position, for example, can directly or indirectly score the nucleotide change or changes. For example, allele-specific primer extension assays can employ detection positions at the probe's terminus as exemplified in FIG. 2. In other embodiments, single base extension assays can detect an allele at a position adjacent to the probe's terminus as exemplified in FIG. 1. Other exemplary nucleic acid detection methods which can detect SNPs based on target-specific modification of one or more probes include, for example, ligation, primer extension followed by ligation, and nucleotide sequencing.

In some embodiments of the invention, probes are designed for detection of allelic variants in genes or in their corresponding transcripts. For example, target specific probes can be designed to detect any of the common biallelic SNPs occurring at a particular nucleotide position. Such common biallelic SNP classes include, for example, [A/T], [C/G], [A/C], [A/G], [T/C] and [T/G], where the two nucleotides within brackets represent the alternative SNP nucleotides that constitute two different alleles of the same gene. Probes for other biallelic loci also can be designed and used in the compositions and methods of the invention. Similarly, probes for triallelic and tetraallelic loci also can be designed and utilized in the compositions and methods of the invention.

Triallelic loci can be distinguished, for example, using the probe extension assay shown in FIG. 2 modified to include a set of three bead types for each locus instead of only two bead types used for detection of biallelic loci. Thus, each allele would be targeted, respectively, by one of three probes present on different beads such that a sample that is homozygous for a single allele would produce signal indicative of a particular label bound to one of the beads and a sample that was heterozygous for all three alleles would produce signal indicative of particular labels bound to all three of the beads. Similarly, tetralleleic loci can be distinguished using four bead types in the assay exemplified in FIG. 2. Although detection of triallelic and tetraallelic loci is exemplified with respect to FIG. 2, it will be understood that other detection platforms and assay components can be used in a similar fashion.

With reference to the biallelic SNP [A/G] for exemplification, target specific probes can be designed for single nucleotide detection to occur, for example, at the SNP or following the SNP. For example, detection formats using enzymatic modification, such as polymerase extension in sequencing reactions, in extension-ligation reactions or in single base extension reactions, can be employed as a SNP detection method. One particularly useful probe design for this type of detection assay can include complementarity to a region of the target that is 3′ to the SNP. Thus, the region of the probe that hybridizes to the target would be 5′ to the SNP detection position and the 3′ end of the probe would be available for target-specific modification. Hybridization of the same probe to all alleles present in the mixture followed by enzymatic extension using each of four nucleoside triphosphates (NTP) containing distinguishable labels will result in incorporation of labels indicative of the SNP into the extension product. For example, employing a red fluorescent label attached to T nucleotides and a green fluorescent label attached to C nucleotides will result in the incorporation of red signal in the probe for the A allele and green detectible signal in the probe for the G allele. Continuing with this example, where a [T/C] biallelic locus is to also be detected in this format, a single probe can be used for T and C detection by using A and G nucleoside triphosphates containing labels that are distinguishable from each other and also distinguishable from the red and green labels attached to the T and C nucleotides. In this particular probe/detection method format combination, designing the detection position immediately adjacent to the terminus of the target specific probe is particularly useful because it will reduce incorporation of signal by labeled nucleotides at positions other than the detection position.

In other exemplary detection formats, target specific probes are designed to contain the detection position internal to or at the terminus of the probe. For example, detection formats utilizing enzymatic activities such as polymerase extension or nucleic acid ligation can be designed to require the terminal nucleotide of the target specific probe to be complementary and hybridized to its target nucleic acid in order for enzymatic modification to occur. In these specific formats, [A/G] specific probes can be designed to contain a terminal T on one probe specific for the A allele and a terminal C on a second probe specific for the G allele. Inclusion of these T and G containing probes into a multiplex detection method of the invention employing, for example, polymerase extension, will incorporate adjacent nucleotides as extension products where correct hybridization occurs between the 3′ terminal nucleotide of the probe and the target nucleic acid. Accordingly, in this probe design, exemplified in FIG. 2, the allelic detection position contained within the target specific probe and the label is incorporated as an extension product under conditions of terminal nucleotide complementarity. Indicative labels for this probe/detection method format combination should distinguish between label incorporation at the adjacent nucleotides of different probes.

The different probes can be included on the same multiplex substrate element or on different elements so long as signal, location or both can be distinguished between the different assayed alleles. Once the target specific probes are designed or selected they are attached to a multiplex substrate element of the invention.

Attachment can occur by any of a variety of methods well known to those skilled in the art including, for example, chemical, photochemical, photolithography, enzymatic and/or affinity binding. Specific examples of methods used for attachment have been exemplified previously with reference to nucleic acids attached to arrays or microspheres. Other methods well known to those skilled in the art also can be employed.

The target specific probes also can be attached to a multiplex substrate element in a variety of different configurations. Particularly useful embodiments of the invention employ at least two different target specific probes attached to a substrate element. The level of multiplexing can be increased according to need or preference to contain more than two different target specific probe per substrate element. For example, four or more different target specific probes can be attached to a single substrate element. Attachment of four or more target specific probes will allow detection of four different analytes employing a single substrate element. Similarly, using a population of substrate element having four or more attached target specific probes will allow detection of twice as many analytes employing the same number of substrate elements having only two different attached probes. Therefore, multiplex substrate elements of the invention can have, for example, 2, 3, 4, 5, 6, 7, 8, 9, 10, 11, 12, 13, 14, 15, 16, 17, 18, 19 or 20 or more different target specific probes attached to a single element. In some specific embodiments, the multiplex level can be greater than 20 different target specific probes attached to a single substrate element and include, for example, 21, 22, 23, 24, 25, 26, 27, 28, 29, 30, 35, 40, 45 or 50 or more different probe sequences. Following the teachings and guidance provided herein, those skilled in the art will understand that the level of multiplexing can be selected according to the user's preferences and can include factors such as number of samples evaluated, number of determinations per sample and/or available assay time.

Similarly, a particularly useful embodiment of the invention employs a single identifier sequence per substrate element type. The single identifier identifies both the location of the element within an array and the at least two different target specific probes attached to the element. However, as with the number of different target specific probes attached to a substrate element, the number of different and unique identifier sequences also can vary depending, for example, on the intended use and level of multiplexing of the detection format. Accordingly, a substrate element can have, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45 or 50 or more different identifier sequences attached to its surface. They can be single identifier sequences or bi-, tri- and/or multipartite structures and some or all of the identifier sequences can be linked to a target specific probe or exist as separate entity attached to the element. Therefore, each identifier sequence also can have a number of different subregions including, for example, 2, 3, 4, 5, 6, 7, 8, 9, 10, 11, 12, 13, 14, 15, 16, 17, 18, 19 or 20 or more different portions.

When the multiplexing level of target specific probes increases per substrate element, a particularly useful means of identifying both the substrate element and some or all of its associated target specific probes is to include multiple unique identifier sequences in order to further decipher some or all of the attached target specific probes. For example, including a one-to-one correspondence between identifier sequence, or subregion of an identifier sequence, to target specific probe will provide a one-to-one correspondence between identifier and probe, allowing for quick and efficient decoding of the analyte, probe and substrate element location. All other combinations and permutations also can be employed for single and/or multi-step deconvolution of groupings of target specific probes into identifiable species. Decoding and deconvolution of complex signals are well known in the art. Given the teachings and guidance provided herein, those skilled in the art will understand that a variety of different configurations can equally be employed in the compositions and methods of the invention to achieve a desired number of decoding steps given the level of multiplexing used on one or more substrate elements of the invention.

In the specific embodiment of target nucleic acid detection, the multiplex substrate elements of the invention are employed in hybridization-based detection and identification steps. Target specific probes hybridize to targets and can be isolated, for example, prior to detection or nucleotide sequence determination. Alternatively, detection and/or nucleotide sequence determination can be performed without prior isolation of the hybridized complexes. Similarly, following or simultaneously to detection or sequence determination, the identifier sequences are hybridized to complementary decoder sequence for identification of substrate element type and location. Briefly, target specific probes and identifier sequences are contacted with a target containing sample under conditions sufficient for hybridization and the hybridization complexes can be separated from unhybridized nucleic acid by washing, for example. The greater the specificity of a target specific probe or identifier sequence for its target or complementary sequence, respectively, within a sample containing a mixture of targets or complementary decoders the greater the accuracy that can be achieved in the detection result.

A variety of hybridization or washing conditions can be used in the target nucleic acid detection methods of the invention. Hybridization or washing conditions are well known in the art and can be found described in, for example, Sambrook et al., Molecular Cloning: A Laboratory Manual, Third Ed., Cold Spring Harbor Laboratory, New York (2001) and in Ausubel et al., Current Protocols in Molecular Biology, John Wiley and Sons, Baltimore, Md. (1999). Stringency of the hybridization or washing conditions include variations in temperature or buffer composition and can be varied according to the specificity of the reaction needed. A range of stringency includes, for example, high, moderate or low stringency conditions.

Stringent conditions include sequence-dependent specificity and will differ according to length and content of target and probe nucleic acids. Longer sequences hybridize more specifically at higher temperatures. Generally, stringent conditions are selected to be about 5-10° C. lower than the thermal melting point (Tₘ) for the specific sequence at a defined ionic strength and pH. The Tₘ is the temperature, under defined ionic strength, pH and nucleic acid concentration, at which 50% of the probes complementary to the target hybridize to the target sequence at equilibrium. Differences in the number of hydrogen bonds as a function of base pairing between perfect matches and mismatches can be exploited as a result of their different Tₘs. Accordingly, a hybrid including perfect complementarity will melt at a higher temperature than one including at least one mismatch, all other parameters being equal.

Stringent hybridization conditions also include those in which the salt concentration is less than about 1.0 M sodium ion, generally about 0.01 to 1.0 M sodium ion concentration or other salts at pH 7.0 to 8.3 and the temperature is at least about 30° C. for short probes such as 10 to 50 nucleotides and at least about 60° C. for long probes such as greater than 50 nucleotides. Low stringency conditions include NaCl concentrations of about 1.0 M. Furthermore, low stringency conditions can include MgCl₂ concentrations of about 10 mM, moderate stringency of about 1-10 mM, and high stringency conditions include concentrations of about 1 mM. Stringent conditions also can be achieved with the addition of helix destabilizing agents such as formamide. For example, low stringency conditions include formamide concentrations of about 0 to 10%, while high stringency conditions utilize formamide concentrations of about 40%. For a further description of hybridization conditions and its relationship to stringency see, for example, Tijssen, Techniques in Biochemistry and Molecular Biology—Hybridization with Nucleic Acid Probes, Overview of principles of hybridization and the strategy of nucleic acid assays. (1993).

The multiplex substrate elements of the invention can be produced on an as needed basis or, alternatively, they can be produced and stored for later employment in a detection method of the invention. Similarly, as will be apparent from the teachings and guidance provided below with respect to the methods of the invention, a substrate element or a population of substrate element complexes having hybridized or bound target analytes also can be produced using the methods of the invention and stored for later analysis and/or detection. In this specific embodiment, unbound targets can be, for example, removed following hybridization and some or all of the hybridized complexes can be stored for later determinations. Alternatively, the hybridized or bound substrate element complexes can be stored without a wash step. Storage can involve short or long periods of time depending on the user's preferences. For example, storage can be, for example, for the time needed to complete other multiplex assays within a particular analysis or for longer periods of time including, for example, days, weeks, months or years. Storage conditions suitable for the type of analyte are sufficient to maintain stability of the complexes prior to subsequent use. Such conditions include, for example, room temperature, 4° C., −20 ° C. and −70 ° C.

In addition to isolation and/or storage of a multiplex substrate element or a population of different types of multiplex substrate elements prior to hybridization, the elements also can be isolated for analysis, later use and/or storage following use in any of the detection procedures exemplified herein or well known in the art. Isolation of elements at this stage in a detection method of the invention will result in the separation of substrate element complexes which also have labels incorporated into the target molecule indicative of that particular analyte. For example, a substrate element hybridization complex or population of different complexes employed in the detection of a target nucleic acid analyte can be input into a nucleic acid detection method of the invention where targets or target nucleotide sequences are distinguished through incorporation of distinct labels into the target or at a particular detection position in the target.

In a particularly useful embodiment, distinguishing labels can emit distinguishing signals having different spectral wavelengths. For example, A can emit a red signal, C a green signal T a yellow signal and G a blue signal. Incorporation of one of these exemplary labels at a detection position will result in different complexes within the population having different labels incorporated into the complexed target nucleic acid and indicative of the target molecule and/or the nucleotide sequence of interest in the target molecule. For the specific embodiment of single nucleotide polymorphism detection, a target molecule incorporating an A at the detection position will result in a substrate element hybridized to its respective target nucleic acid in a complex which has an A in the detection position having an attached indicative red label. Within the same population of complexed substrate elements, a target molecule incorporating a C at the detection position will result in a substrate element hybridized to its respective target nucleic acid in a complex which has a C in the detection position having an attached indicative green label. Similarly, other substrate elements within the same population of complexes will contain target molecules incorporating T or G at their respective detection positions will result in a substrate element hybridized to their target nucleic acids and containing a T or G in their detection positions respectively having an attached indicative yellow or blue label.

A variety of populations can be obtained or isolated depending on the structure and format of the detection assay and target specific probes and the labels employed for distinguishing detection positions. Accordingly, the embodiment described above is exemplary. Those skilled in the art will understand that red, green, yellow and blue emitting labels can be substituted with any of a variety of other distinguishing labels well known in the art. Moreover, the label management for distinguishing target nucleic acid determination or nucleotide sequence detection can be equally modified according to the need of the user and other indicative features for distinguishing target nucleic acid. Therefore, the separated or isolated substrate element-target complexes can include, for example, two, three or four or more indicative labels. Furthermore, the labels can be incorporated into nucleotides used to modify probes in the presence of a specific target as exemplified above or the labels can be present as modifications of the targets that are to be detected.

Therefore, the invention provides a multiplex substrate element, having an attached first nucleic acid and an attached second nucleic acid, the first nucleic acid including a first target specific probe, a hybridized first target nucleic acid and a first nucleotide having a first label indicative of the first target nucleic acid, the attached second nucleic acid includes a second target specific probe, a hybridized second target nucleic acid and a second nucleotide having a second label indicative of the second target nucleic acid, wherein the first target nucleic acid has a sequence that is different from the second target nucleic acid, and wherein the first label is distinctive from the second label. The multiplex substrate element also can include one or more attached identifier sequences.

The invention also provides a population of modified target specific probes having a plurality of different multiplex substrate elements, each element including an attached first nucleic acid and an attached second nucleic acid, the first nucleic acid including a first target specific probe, a hybridized first target nucleic acid and a first nucleotide having a first label indicative of the first target nucleic acid, the attached second nucleic acid including a second target specific probe, a hybridized second target nucleic acid and a second nucleotide having a second label indicative of the second target nucleic acid, wherein the first target nucleic acid has a sequence that is different from the second target nucleic acid, and wherein the first label is distinctive from the second label. Each multiplex substrate element within the population also can include one or more attached identifier sequences. The multiplex substrate elements also can contain attached

The invention further provides a method of detecting nucleic acid sequences. The method includes: (a) contacting under conditions sufficient for hybridization a population of target nucleic acids with a plurality of multiplex substrate elements, each element including an attached first nucleic acid and an attached second nucleic acid, the first nucleic acid including a first target specific probe, the second nucleic acid including a second target specific probe, thereby forming hybridization complexes including the first target specific probe with a first target nucleic acid and the second target specific probe with a second target nucleic acid, wherein the first target nucleic acid has a sequence that is different from the second target nucleic acid; (b) contacting the hybridization complexes with a polymerase and a nucleotide mixture to modify at least one of the target specific probes, thereby forming at least one modified target specific probe, the nucleotide mixture containing at least two nucleotides having first and second distinct labels, respectively, and (c) determining incorporation of the first or second label into the at least one modified target specific probe, thereby determining the presence or absence of the first or second target sequences.

The methods of the invention employ the multiplex substrate elements of the invention to judicially reduce the substrate element requirements for any particular set of measurements while concomitantly increasing the number of possible determinations that can be achieved in any given assay. The multiplex capability of the substrate elements allow for efficient and simultaneous detection of many different target nucleic acids on the same element as well as across many different elements in the same assay. The modularity in the compositions and methods of the invention complement the multiplex detection capability per substrate element and per assay because they can be used in conjunction with a label management scheme of the invention to detect a vast number of different target nucleic acids simultaneously in the same array or multiplex scheme.

The multiplex detection methods include contacting a population of target nucleic acids with a plurality of multiplex substrate elements. Conditions sufficient for hybridization include those described previously such as appropriate Tₘ of target specific probes, GC content of target specific probes, temperature and salt concentration as well as other conditions well known in the art. Given the predetermined composition of target specific probes in that they can be, for example, designed and/or selected to hybridize to known target nucleic acids, the sequence of the probe and target generally will be known. Those skilled in the art will know, or can readily determine by, for example, calculation or empirically testing, the hybridization specificity of any particular target specific probe or of a population of probes in general. Similarly, given the teachings and guidance provided herein, including that which is well known in the art of hybridization, those skilled in the art can readily design and/or select a particular probe, probe pair, probe set or a population of probes for some or all of a multiplex assay to hybridize specifically under a predetermined set of conditions. Accordingly, conditions sufficient for hybridization of target specific probes with target nucleic acids generally will be, for example, predetermined or known at the time of probe design. Target specific probes are contacted for a sufficient period of time given the hybridization conditions to form hybridization complexes between attached first, second, third and/or fourth or more target specific probes attached to each substrate element with any of their complementary target nucleic acids contained in the sample. Thus, targets of known composition can be detected in a sample to determine whether or not they are present in the sample or to determine the amount of each target present in the sample.

In some embodiments of the invention, each multiplex substrate element is attached to at least a first target specific probe and a different second target specific probe. Various alternative substrate element and target specific probe structures, compositions and quantity of different attached target specific probes have been exemplified previously. Any of these formats or configurations can be employed in the methods of the invention. The at least first and second attached target specific probes are used for nucleic acid detection and/or nucleotide sequence detection or determination through hybridization to their complementary target nucleic acids within a sample followed by employment of the hybridized complexes in a detection assay. Accordingly, following hybridization to a sample containing or suspected of containing the target nucleic acids of interest the attached first and second target specific probes, for example, will form hybridization complexes with their respective first and second target nucleic acids when present in a sample.

Samples applicable for assessing the presence or absence of an analyte, for assessing one or more characteristics of an analyte present as a component in a sample have been exemplified previously. Briefly, samples include any of a variety of isolated, partially purified or crude mixtures of molecules obtained from biological sources. Such sources include, for example, genomic and other DNA populations, RNA populations, polypeptide populations and populations of carbohydrate, lipid and other macromolecules as well as small molecules. Samples containing such component analytes can be obtained from sources using methods well known in the art. Exemplary sources include, for example, eukaryotic and/or mammalian tissues, bodily fluids, cells or nucleic acids, including human, prokaryotic cells or nucleic acids and/or plant tissue, cells or nucleic acid as exemplified previously.

Once samples containing or suspected of containing target analytes have been contacted with a population of multiplex substrate elements and hybridization complexes formed, for example, various steps can be performed prior to detection analysis. For example, unbound targets can be removed from the hybridization complexes. Similarly, in the specific example where the analyte is a polypeptide, uncomplexed targets can also be removed from the mixture. Procedures to remove unbound analytes from, for example, a hybridization complex or an affinity complex, are well known in the art and include, for example, washing, liquid-liquid extraction, solid-phase extraction, centrifugation of attached solid supports, precipitation, magnetic force using magnetic solid supports and enzymatic or chemical digestion. Various other methods well known in the art can similarly be used for separation or removal of bound analyte complexes from unbound, free target nucleic acids.

Employing the multiplex substrate elements and methods of the invention, the population of hybridization complexes is subjected to any of a variety of analyte detection methods. For the specific embodiment of nucleic acid detection, particularly useful detection methods employ modifying the probe in a target-specific fashion using the target as a template and a nucleic acid template directed enzyme. Such enzymes include, for example, DNA or RNA directed polymerases and ligases. For purposes of illustration, the multiplex detection methods of the invention are described below with reference to enzymatic incorporation of detectable nucleotides into a probe using polymerase. Various alternative template-directed or other enzymatic detection methods are described elsewhere below for the further exemplification of the variety of detection methods applicable to use with the multiplex substrate elements and methods of the invention.

Extension assays are particularly useful for nucleic acid detection and/or nucleotide determination. Extension assays are generally carried out by modifying the 3′ end of a probe nucleic acid when hybridized to its complementary target nucleic acid. In this configuration, the probe nucleic acid functions as a primer for polymerase extension. The target nucleic acid can act as a template directing the type of modification, for example, by base pairing interactions that occur during po 1 ymerase-based extension of the probe nucleic acid to incorporate one or more nucleotides. Polymerase extension assays are particularly useful, for example, due to the relative high-fidelity of polymerases and their relative ease of implementation. Extension assays can be carried out to modify nucleic acid probes that have free 3′ ends, for example, when bound to a substrate element such as an arrayed population of multiplex substrate elements of the invention.

The population of hybridization complexes is contacted with a polymerase and a nucleotide mixture for incorporation of one or more detectable nucleotides at a detection position. For example, in the specific example of SNP detection for correlation of the presence or absence of alleles associated with a pathological condition, allele specific primer extension, single base extension or single base sequencing are particularly useful extension assays for determining the polymorphic nucleotide at the detection position.

In particular embodiments of the invention, single base extension (SBE) can be used for target nucleic acid detection or nucleotide determination in a target nucleic acid. SBE is exemplified in FIG. 1 using the multiplex substrate elements of the invention. This extension method utilizes an extension target specific probe that hybridizes to a target nucleic acid at a location that is proximal or adjacent to a detection position, the detection position being indicative of a particular sequence. A polymerase can be used to extend the 3′ end of the probe with a nucleotide analog labeled with a detection label. Based on the fidelity of the enzyme, a nucleotide is only incorporated into the extension probe if it is complementary to the detection position in the target nucleic acid. If desired, the nucleotide can be derivatized such that no further extensions can occur, and thus only a single nucleotide is added. The presence of the labeled nucleotide in the extended probe can be detected, for example, at a particular location in an array and the added nucleotide identified to determine the identity of the analyte sequence. SBE can be carried out under known conditions such as those described in U.S. patent application Ser. No. 09/425,633. A labeled nucleotide can be detected using methods such as those set forth above or below, or as described elsewhere such as in Syvanen et al., Genomics 8:684-692 (1990); Syvanen et al., Human Mutation 3:172- 179 (1994); U.S. Pat. Nos. 5,846,710 and 5,888,819; Pastinen et al., Genomics Res. 7(6):606-614 (1997).

In an alternative embodiment, single base sequencing can be employed for target nucleic acid detection or nucleotide determination in a target nucleic acid. Single base sequencing (SBS) is an extension assay that can be carried out as set forth above for SBE with the exception that one or more non-chain terminating nucleotides are included in the extension reaction. Thus, in accordance with the invention, one or more non-chain terminating nucleotides can be included in an SBE reaction including, for example, those exemplified above.

ASPE is an extension assay that utilizes extension probes that differ in nucleotide composition at their 3′ end. ASPE is exemplified in FIG. 2 using multiplex substrate elements of the invention. This extension method can be carried out by hybridizing a target nucleic acid to a target specific extension probe having a 3′ sequence portion that is complementary to a detection position and a 5′ portion that is complementary to a sequence that is adjacent to the detection position. Template directed modification of the 3′ portion of the probe, for example, by addition of a labeled nucleotide by a polymerase yields a labeled extension product when the template includes the hybridized target nucleic acid. The presence of such a labeled primer-extension product can then be detected, for example, based on its signal and/or location in an arrayed population of multiplex elements to indicate the presence of a particular analyte or sequence. If desired, the nucleotide used in an ASPE reaction can be derivatized such that no further extensions can occur, and thus only a single nucleotide is added. This format is referred to as allele-specific single base extension (ASSBE).

In particular embodiments, ASPE can be carried out with multiple extension probes that have similar 5′ ends such that they anneal adjacent to the same detection position in a target nucleic acid but different 3′ ends, such that only probes having a 3′ end that complements the detection position are modified by a polymerase. For example, a target specific probe having a 3′ terminal base that is complementary to a particular detection position is referred to as a perfect match (PM) probe for the position, whereas probes that have a 3′ terminal mismatch base and are not capable of being extended in an ASPE reaction are mismatch (MM) probes for the position. In the multiplex example illustrated in FIG. 2, for example, probe 4 is shown as a mismatch while target specific probes 1, 2 and 3 are shown as a perfect match.

The presence of the labeled nucleotide in the PM probe can be detected and the 3′ sequence of the probe determined to identify a particular analyte sequence. An ASPE reaction can include 1, 2, or 3 different MM probes, for example, at discrete array locations, the number being chosen depending upon the diversity occurring at the particular locus being assayed. For example, two probes can be used to determine which of two alleles for a particular locus are present in a sample, whereas three different probes can be used to distinguish the alleles of a 3-allele locus. In particular embodiments, an ASPE reaction can include a nucleotide analog that is derivatized to be chain terminating. Thus, a PM target specific probe in a probe-fragment hybrid can be modified to incorporate a single nucleotide analog without further extension. Although primer extension methods are exemplified herein with regard to modification of a substrate-attached probe when hybridized to a target, it will be understood that the same principles can be applied in the case where the 3′ end of the hybridized target is modified using the substrate-attached probe as the template.

FIGS. 1 and 2 schematically exemplify the use of colored labels where each color corresponds to a different signal that is distinguishable from the other colored signals in a multiplex mixture. The signals can include, for example, optical signals such as fluorescent or luminescent signals as described above. Multiplex detection of one or more target nucleic acids within a population using the methods of the invention couples the assay format and probe configuration with use of distinguishable labels attached or attachable to a nucleotide indicative of the detection position. In FIGS. 1 and 2, the different colors exemplify different fluorescent probes that emit different and distinguishable wavelengths. For example, FIG. 1 illustrates blue (B), yellow (Y), red (R) and green (G) colored labels corresponding to emission wavelengths within the blue, yellow, red and green regions, respectively, of the electromagnetic spectrum. Each of these emission wavelengths are sufficiently different to be distinguishable from each other when combined into a common detection setting using fluorescent detection methods well known in the art. Similarly, given the teachings and guidance provided herein, those skilled in the art will know that any of the other types of labels exemplified above producing different or measurably distinguishable signals also can be selected for use in the methods of the invention. Selection of such other types will be based on factors such as signal distinguishably within a common detection procedure, ease of attachment to nucleotides and stability, for example.

One specific arrangement of probe configuration and usage of distinguishable labels is shown in FIG. 1 where two substrate elements each contain two different target specific probes. The extension assay in this specific embodiment is SBE and scores the nucleotide type at the detection position by incorporation of a labeled nucleotide to the 3′ termini of each of the four probes. Use of the four nucleotides A, T, G and C each differently labeled and distinguishable from the other labeled nucleotide types allows for detection of any of these nucleotide types and identification of the nucleotide and its complement at the detection position.

For example, FIG. 1 illustrates one multiplex substrate element (denoted as the upper bead type 1) containing probes 1 and 2 (purple and blue, respectively), each constituting a different sequence. For purposes of illustration, a second substrate element (lower) is shown having an identical pair of first and second probes. Each probe is locus specific such that it can bind all alleles but different target nucleic acids can be distinguished because the nucleotide at the detection position differs. Typically each bead will have multiple copies of each probe such that a single bead will be labeled with all four nucleotides shown in FIG. 1 if the sample is heterozygous for both loci (i.e. the sample contains both alleles of both loci). This probe and detection format is particularly useful for detecting different allelic variants of the same gene by detecting one or more nucleotide polymorphisms at the detection position.

Probe 1 in the upper substrate element of FIG. 1 detects an allele containing a T at the detection position by incorporation of an A labeled with a red signal. In comparison, probe 1 attached to the lower substrate element detects an allele containing an A at the detection position by incorporation of a T labeled with a yellow signal. Similarly, with respect to the other probes on the beads, probe 2 on the upper element detects an allele containing a G at the detection position by incorporation of a C labeled with a green signal. Probe 2, attached to the lower substrate element, as illustrated in FIG. 1 detects the G allele of the same locus. FIG. 1 therefore exemplifies that the same target specific probe can be used to detect multiple different nucleotides at one or more detection positions when used in combination with differentially labeled nucleotides.

FIG. 1 illustrates the incorporation of different nucleotide types at the same detection position for nucleic acid detection and/or nucleotide sequence determination between different target nucleic acids. A total of two different target specific probes are illustrated to detect three different target nucleic acids (probe 1 detects the T and A alleles of a first locus and probe 2 detects the C allele of a second locus). Employing the same two target specific probes also can detect any of the four different alleles for each of gene A and gene B through incorporation and detection of an indicative nucleotide having a distinct label. For example, a plurality of probe 1 attached to different multiplex substrate elements can hybridize to alleles 1, 2, 3 and 4 of gene A. Incorporation of a G labeled with a blue signal identifies a C at the detection position for allele 1, for example. Incorporation of a C labeled with a green signal identifies a G at the detection position for allele 2, for example. Incorporation of a T labeled with a yellow signal identifies an A at the detection position for allele 3 whereas incorporation an A labeled with a red signal identifies a T at the detection position for allele 4, for example.

Given the teachings and guidance provided herein, those skilled in the art will understand that, for example, an SBE probe configuration or similar probe configurations for other extension methods can be employed to achieve detection of all variants at a detection position employing different nucleotide types having distinct labels. Detection of the distinct label identifies the labeled nucleotide type and its complement at the detection position. Similarly, employing the multiplex substrate elements, label usage, detection method and probe designs as exemplified herein, the methods of the invention allow for a large number of nucleic acid determinations in a single assay. For example, a plurality of multiplex substrate elements can be used with a mixture of all four nucleotide types each being distinctly labeled. Each substrate element can have two, three or four or more different target specific probes. Identification of the presence or absence of a target nucleic acid and/or of the nucleotide sequence at a detection position can be determined using, for example, an SBE extension method and determining which type of the labeled nucleotides are incorporated at the detection position.

Another specific arrangement of probe configuration and usage of distinguishable labels is shown in FIG. 2 where two different types of substrate elements are illustrated. Each contains two different target specific probes that also differ from the two probes attached to the other substrate element. The extension assay illustrated in this specific embodiment is ASPE and scores the nucleotide type at the detection position by incorporation of a labeled nucleotide adjacent to the detection position. Hence, for ASPE, the 3′ terminus of each probe corresponds to the detection position. In this specific exemplification, two distinct labels are used in conjunction with all four nucleotides A, T, G and C. A and T are similarly labeled (red; R) as are G and C (green; G). For biallelic determination, scoring the SNP at the detection position is based on incorporation of label adjacent to the detection and assessment of the relative amount of label incorporated into probes for allelic variants on separate substrate elements.

For example, FIG. 2 illustrates one multiplex substrate element (denoted as bead type 1) containing probes 1 and 2 (purple and blue, respectively), each constituting a different sequence. The second substrate element (denoted as bead type 2) contains probes 3 and 4 (yellow and green, respectively) which differ in sequence compared to each other and compared to probes 1 and 2. Each of probes 1 and 3 score a different nucleotide allele at the detection position (G and C, respectively) of the same locus, but incorporate the same labeled nucleotide adjacent thereto since the target contains a T at this position. Probe 2 is illustrated in FIG. 2 to score a G nucleotide at the detection position by incorporation of an adjacent C, whereas no nucleotide is scored at probe 4 indicating absence of the allele having a C allele at the respective locus. Thus, the beads shown in FIG. 2 have scored a G/C heterozygote at the locus targeted by probes 1 and 3 and have also scored a G homozygote at the locus targeted by probes 2 and 4. In this configuration, determining the presence or absence of a label adjacent to the detection position of a target specific probe identifies the target nucleic acid and/or one or more polymorphic sequences. As with SBE, this ASPE-based probe and detection format also is particularly useful for detecting different allelic variants of the same gene by detecting one or more nucleotide polymorphisms at the detection position. FIG. 2 therefore exemplifies that extension assays using ASPE or other similar format employ different target specific probe to detect different target analytes or monomer types therein at one or more detection positions when used in combination with at least two distinct labels such as the two pairs of differentially labeled nucleotides exemplified above.

Given the teachings and guidance provided herein, those skilled in the art will understand that, for example, an ASPE probe configuration or similar probe configurations for other extension methods can be employed to achieve detection of all variants at a detection position employing different nucleotide types having subsets of distinct labels. Detection of the distinct label within a subset identifies the labeled nucleotide type and its complement at, for example, an adjacent detection position. As with the SBE and four label combination exemplified above, those skilled in the art will understand that sets of labels which distinguish subsets of nucleotide types (e.g., A and T from G and C) similarly can be employed using the multiplex substrate elements and methods of the invention for determination of a large number of different target nucleic acids in a single assay. For example, a plurality of multiplex substrate elements can be used with a mixture of all four nucleotide types where at least two are distinctly labeled. Each substrate element can have two, three or four or more different target specific probes. Identification of the presence or absence of a target nucleic acid and/or of the nucleotide sequence at a detection position can be determined using, for example, an ASPE extension method and determining which type of the labeled nucleotides are incorporated at the detection position.

As exemplified above and previously with respect to the modular multiplex capabilities of the multiplex substrate elements of the invention, the methods of the invention can be used for the detection of a wide range of population sizes for analytes such as target nucleic acids. Population sizes include, for example, from two or more analytes to greater than 10⁶ or 10⁷. Useful population sizes for detection and/or sequence determination of its constituent analytes include, for example, 10, 25, 50, 75, 100, 200, 300, 400, 500, 750, 1000, 2000, 3000, 4000, 5000, 6000, 7000, 8000, 9000 or 10,000 or more analytes in a single assay or determination. Other particularly useful populations include, for example, 10⁵, 10⁶, 10⁷, 10⁸, 10⁹ or more different target analytes. Population sizes of target analytes corresponding to all numbers above, below or in between these exemplary population sizes also can be employed in the methods of the invention for nucleic acid analysis or detection of some or all of its members. The number of target specific probes employed in these exemplary detections can be the more, less or the same as the number of target analytes depending on, for example, the probe design, detection method and mixture of labels used. The number of multiplex substrate elements employed in these exemplary detections can be, for example, the same or less than the number of target analytes given these same considerations as well as the level of multiplexing employed with each substrate element.

A variety of detectible labels can be used in the methods of the invention to determine the presence or absence of one or more target nucleic acids within a sample population and/or to determine the nucleotide sequence at one or more positions within one or more target nucleic acids within a sample population. Different labels contained in a mixture for concurrent and/or sequential detection are selected to produce distinct signals that can be differentiated in a method of the invention. Distinctness can be accomplished by, for example, employing labels producing the same or different type of signal. For example, a set of labels where all emit fluorescent signals can be employed as the type of label. The signals can be distinguished where each label within the set emits a different colored wavelength. Similarly, a set can include different types of labels where some or all generate different types, and therefore, distinct of signals. For example, a set can be generated where one or more labels are fluorescent and one or more labels are luminescent, reflectance and/or radioactive.

Examples of labels which are useful for detection and which can be combined into a set of distinct labels include, for example, fluorophores, radiolabels, quantum dots, chromophores, enzymes, affinity ligands, electromagnetic spin moieties, heavy atoms, nanoparticle light scattering labels or other nanoparticles or spherical shells and labels having any other signal generation known to those of skill in the art. Specific examples of a variety of fluorescent labels having distinct wavelengths are described further below. Non-limiting examples of label moieties useful for detection in the methods of the invention include, without limitation, suitable enzymes such as horseradish peroxidase, alkaline phosphatase, β-galactosidase and/or acetylcholinesterase; members of a binding pair that are capable of forming complexes such as streptavidin/biotin, avidin/biotin and/or an antigen/antibody complex including, for example, rabbit IgG and anti-rabbit IgG; fluorophores such as umbelliferone, fluorescein, fluorescein isothiocyanate, rhodamine tetramethyl rhodamine, eosin, green fluorescent protein, erythrosin, coumarin, methyl coumarin, pyrene, malachite green, stilbene, lucifer yellow, Cascade Blue™, Texas Red, dichlorotriazinylamine fluorescein, dansyl chloride, phycoerythrin, fluorescent lanthanide complexes such as those including Europium and Terbium, Cy3, Cy5, molecular beacons and fluorescent derivatives thereof, as well as others known in the art as described, for example, in Principles of Fluorescence Spectroscopy, Joseph R. Lakowicz (Editor), Plenum Pub Corp, 2nd edition (July 1999) and the 6th Edition of the Molecular Probes Handbook by Richard P. Hoagland; a luminescent material such as luminol; light scattering or plasmon resonant materials such as gold or silver particles or quantum dots; or radioactive material include ¹⁴C, ¹²³I, ¹²⁴I, ¹²⁵I, ¹³¹I, Tc99m, ³⁵S or ³H.

Particularly useful fluorescent labels for attaching to different nucleotide types, for example, and creating different sets of detection labels include, for example, FAM, Alexa555, Alex 647 and Alexa 750 (all from Invitrogen Corp., San Diego, Calif.). Each of these labels have an emission wavelength distinguishable from the other and therefore, can be used in a common detection mixture for incorporation of different nucleotide types into first, second, third and fourth target nucleic acids. For example, FAM has an excitation wavelength of 488λ and an emission wavelength of 505λ, which is in the visible green light of the electromagnetic spectrum (˜490-540λ). Alexa555 has an excitation wavelength of 555λ and an emission wavelength of 565λ, which is in the red-orange region of the visible light spectrum (˜565-605λ). Alexa647 has an excitation wavelength of 650λ and emits at 668λ in the far-red region of the visible spectrum (˜645-670λ) whereas Alexa750 is excited at 749λ and emits at 775λ in the near-infrared region of the electromagnetic spectrum (˜685-780λ).

Fluorescent labels emitting signals in any region of the visible area of the spectrum other than those exemplified above also can be used in the methods of the invention to generate sets of labels emitting different and distinguishable signals. Such fluorescent labels having emission wavelengths in any of the visible wavelengths of light include, for example, wavelengths ranging from visible violet light having a wavelength at about 400 nm, indigo light having a wavelength of about 445 nm, blue light having a wavelength of about 475 nm, green light having a wavelength of about 510 nm, yellow light having a wavelength of about 570 nm, orange light has a wavelength of about 590 nm, red light has a wavelength of about 650 nm. Other types of labels that generate signals in the non-visible spectrum of the electromagnetic spectrum also can be used and include, for example, signals within wavelengths of the ultraviolet region between about 50-350 nm, other areas of the visible portion between about 350-800 nm, the near-infrared region between about 700-2500 nm, the infrared region between about 800-3000 nm as well as longer and shorter wavelengths.

Particularly useful fluorescent labels having emissions across the visible spectrum include, for example, Alexa fluor Dyes commercially available from Invitrogen (see, for example, the URL probes.invitrogen.com/handbook/tables/0329.html). Labels within this exemplary family include, for example, Alexa350 which emits blue light at 442 nm, Alexa 405 emitting blue light at 421 nm, Alexa430 emitting yellow-green light at 539 nm, Alex488 emitting green light at 519 nm, Alexa500 emitting green light at 525 nm, Alexa 514 emitting yellow-green light at 540 nm, Alexa532 emitting yellow light at 554 nm, Alex546 emitting orange light at 573 nm, Alexa555 emitting red-orange light at 565 nm, Alexa 568 emitting red-orange light at 603 nm, Alexa594 emitting red light at 617 nm, Alexa610 emitting red light at 628 nm, Alexa633 emitting far-red at 647 nm, Alexa635 emitting far-red at 647 nm, Alexa647 emitting far-red light at 668 nm, Alexa680 emitting near-infrared light at 690 nm, Alexa700 emitting near-infrared light at 723 nm and Alexa750 emitting near-infrared light at 775 nm.

Given the teachings and guidance provided herein, those skilled in the art will appreciate that a wide variety of labels can be employed in the compositions and methods of the invention that will achieve resolution and detection of target nucleic acids within a sample population. Labels are selected to generate distinct signals for each target species as described above by, for example, selecting different labels within a mixture to have distinct excitation and emission spectra. Complete separation in excitation and/or emission spectra is one efficient means to achieve sufficient sensitivity for detection of different labels within a mixture. Other methods well known in the art also can be employed using, for example, two or more different labels lacking complete separation in excitation and/or emission spectra. For example, labels having overlapping spectra can be employed in the compositions and methods of the invention in conjunction with spectral filters or other devices that block excitation and/or emission wavelengths within the overlapping region, thus, separating the signals from each of the different probes within a mixture. Selection of labels having narrower excitation and/or emission spectrums also can be employed to, for example, optimize detection sensitivity by increasing the wavelength separation or to enable use of different labels having relatively close excitation and/or emission spectra. One exemplary label type having narrow emission spectra includes , nanocrystals. Characteristics and use of nanocrystals in array formats can be found described in, for example, U.S. Pat. Nos. 6,890,764, 6,544,732 and 6,770,441 to Illumina, Inc.

Given the teachings and guidance provided herein, those skilled in the art also will understand that the characteristics and/or performance of pairs or repertoire of different labels in a mixture employed in a method of the invention can be readily made and tested for separation of excitation spectra, emission, spectra, detection sensitivity, detection accuracy or detection reproducibility or any and all combination of these characteristics, for example. All that is necessary is for one skilled in the art to combine the different candidate labels into a detection sample, measure resultant signals following excitation or other signal stimulus and determine whether the amount of signal from each label correlates with the amount of a known standard. A positive correlation indicates sufficient signal separation to achieve sensitive measurements in a method of the invention.

Labeling can include a signal amplification technique. Signal amplification can be carried out, for example, using streptavidin-phycoerythrin (SAPE) and a biotinylated anti-SAPE antibody. In one embodiment, a three step protocol can be employed in which nucleic acids that have been modified to incorporate biotin are first incubated with streptavidin-phycoerythrin (SAPE), followed by incubation with a biotinylated anti-streptavidin antibody, and finally incubation with SAPE again. This process creates a cascading amplification sandwich since streptavidin has multiple antibody binding sites and the antibody has multiple biotins. Those skilled in the art will recognize from the teaching herein that other receptors such as avidin, modified versions of avidin, or antibodies can be used in an amplification complex and that different labels can be used such as Cy3, Cy5 or others set forth previously herein. Another example of signal amplification uses nucleic acids labeled with a dinitrophenyl (DNP) moiety that can be detected by an antibody that is labeled with a fluorophore. Further exemplary signal amplification techniques and components that can be used in the invention are described, for example, in U.S. Pat No. 6,203,989 B1. Biotin or DNP can be introduced into a nucleic acid using biotin labeled nucleotides or DNP lableled nucleotides, respectively, such as those commercially available from PerkinElmer or Roche.

In some embodiments of the invention, substrate elements and attached target specific probes were exemplified previously to contain identifier sequences. Identifier sequences are particularly useful where the substrate elements are randomly ordered. However, other methods for spatial localization not requiring identifier sequences also can be used in the methods of the invention. For example, beads can be sequentially loaded onto an array such that a first bead type is loaded and located before the next bead type is loaded and the process is repeated until all bead types are loaded. Alternatively, each bead type can be labeled with a different detectable label such that each bead type produces a unique signal indicative of its identity. For example, substrate elements can be labeled with holographic patterns such as those used in the Veracode technology commercially available from Illumina and described for example, in U.S. Pat. No. 7,106,513; US 2006/0118630 or US 2006/0071075, each of which is incorporated herein by reference. Other labels that can be used to distinguish substrate elements from each other include, but are not limited to, quantum dots, various combinations of quantum dots, fluorophores, various combinations of fluorophores, or the like. Therefore, the inclusion of an identifier sequence will be based on factors such as whether the substrate element multiplex scheme is random or ordered, the need and efficiency of other methods known in the art for identifying substrate element location within, for example, a random or ordered array and/or the user's preferences and available resources.

In specific embodiments of the invention, the methods utilize one or more attached identifier sequences. For example, a multiplex substrate element can include the same identifier sequence attached to all target specific probes. Alternatively, a different identifier sequence can be attached to different target specific sequences. For example, a first identifier sequence can be attached to a first target specific probe and a second identifier sequence can be attached to a second target specific probe. In other embodiments having first through fourth target specific probes, a single identifier sequence can be used to decipher all target specific probes. Alternatively, a first identifier sequence can be attached to a first and a second target specific probe and a second identifier sequence can be attached to a third and a fourth target specific probe. Similarly, first through fourth identifier sequences can be each attached to first through fourth target specific probes, respectively. As described previously and further below, the location of any multiplex substrate element can be based on the first identifier sequence, second identifier sequence, third identifier sequence, fourth identifier sequence or subregion thereof or combinations thereof.

Therefore, the invention provides a method of detecting nucleic acid sequences. The method includes: (a) contacting under conditions sufficient for hybridization a population of target nucleic acids with a plurality of multiplex substrate elements including at least first and second multiplex substrate elements; (i) the first element including an attached first nucleic acid and an attached second nucleic acid, the first nucleic acid including a first target specific probe and the second nucleic acid including a second target specific probe; (ii) the second element including a third nucleic acid and a fourth nucleic acid, the third nucleic acid including a third target specific probe and the fourth nucleic acid including a fourth target specific probe, thereby forming hybridization complexes including the first target nucleic acid and the first target specific probe, the second target nucleic acid and the second target specific probe, the third target nucleic acid and the third target specific probe and the fourth target nucleic acid and the fourth target specific probe; (b) contacting the hybridization complexes with a polymerase and a nucleotide mixture to modify at least one of the target specific probes attached to the first multiplex substrate element and to modify at least one of the target specific probes attached to the second multiplex substrate element, thereby forming at least two modified target specific probes, the nucleotide mixture containing at least two nucleotides having first and second distinct labels, respectively, and (c) determining incorporation of the first or second labels into the modified target specific probes, thereby determining the presence or absence of the first, second, third or fourth target sequences.

The method also can include configurations where the attached first nucleic acid and the attached second nucleic acid each further include a first identifier sequence and wherein the attached third nucleic acid and the attached fourth nucleic acid each further include a second identifier sequence that is different from the first identifier sequence. The first element can be located within the plurality of multiplex substrate elements based on the presence of the first identifier sequence and the second element is located in the plurality of multiplex substrate elements based on the presence of the second identifier sequence. Further, the attached first nucleic acid can further include a first identifier sequence, the attached second nucleic acid further includes a second identifier sequence, the attached third nucleic acid further includes a third identifier sequence and the attached fourth nucleic acid further includes a fourth identifier sequence. The first element can be located within the plurality of multiplex substrate elements based on the presence of the first and second identifier sequences and the second element is located in the plurality of multiplex substrate elements based on the presence of the third and fourth identifier sequences.

Also included is a method of detection where step (b), recited above, further includes contacting the hybridization complexes with a polymerase and a nucleotide mixture to modify at least one of the target specific probes attached to the first multiplex substrate element and to modify at least one of the target specific probes attached to the second multiplex substrate element, thereby forming at least two modified target specific probes, the nucleotide mixture containing a first and second type of nucleotides having a first label and a third and fourth type of nucleotides having a second label, wherein the first and second label are distinguishable from each other and wherein all four types of nucleotide are different from each other. The first target specific probe can hybridize to a first allele of a first locus and the third target specific probe can hybridize to a different allele of the first locus, and the second target specific probe can hybridize to a first allele of a second locus and the fourth probe can hybridize to a different allele of the second locus. Further, the sequence of the first allele can be identified by distinguishing presence or absence of the first signal at the first and second multiplex element and the sequence of the second allele is identified by distinguishing presence or absence of the second signal at the first and second multiplex element.

Further included is a method of detection where step (b), recited above, further includes contacting the hybridization complexes with a polymerase and a nucleotide mixture to modify both of the target specific probes attached to the first multiplex substrate element and to modify both of the target specific probes attached to the second multiplex substrate element, thereby forming four modified target specific probes, the nucleotide mixture containing four types of nucleotides each with a different label, wherein the labels are distinguishable from each other and wherein all four types of nucleotide are different from each other. The first target specific probe and the third target specific probe can have a sequence that hybridizes to two different alleles of a first locus, and wherein the second target specific probe and the fourth target specific probe have a sequence that hybridizes to two different alleles of a different locus. Further, the sequence of each the allele is identified by distinguishing the type of signal present at the first and second multiplex element.

The extension methods exemplified above for detection of a target or a target sequence can be employed in any of the various forms of the methods of the invention. In addition to these extension methods, various other methods well known in the art also can be employed in the methods of the invention. Exemplary embodiments of these various other methods are set forth below for purposes of illustration. All of these exemplary methods are well known in the art and are equally applicable for use in conjunction with the multiplex substrate elements and methods of the invention. Similarly, these and/or other well known procedures also can be combined in various formats and configurations to achieve essentially any desired analysis of a target analyte of the invention. Given the teachings and guidance provided herein, those skilled in the art will understand that the compositions and methods of the invention can be employed in a variety of different procedures to obtain a sought after result. All of such procedures and formats for nucleic acid detection or analysis are well known to those skilled in the art and can be found described in, for example, WO 2005/003304 A2 and in U.S. Patent Application Publications 20050181394, 20050059048, 20050053980, 20050037393, 20040259106, 20040259100.

A target nucleic acid sample can be amplified prior, during or after to hybridization and nucleic acid analysis or detection. Particularly useful methods include, for example, PCR or random primer amplification or other methods described in US 2005/0181394, which is incorporated herein by reference. However, amplification need not be carried out if the sample provides sufficient quantity to suit the particular method being used. A nucleic acid sample for target analysis or detection also can be attached to a solid phase using methods and substrates described elsewhere herein or otherwise known in the art. The sample will typically be attached as a population of separate nucleic acids, such as those encoding genome fragments, that can be distinguished from each other. Microarrays are particularly useful for sequence analysis.

A further analysis or detection method that can be used in conjunction with the compositions and methods of the invention includes, for example, gene expression analysis, methylation analysis and allele-specific expression (ASE) analysis. In particular, methods for on-array labeling of probe nucleic acids using primer extension methods can be used in the detection of RNA or cDNA for such expressed sequence determinations. Probe-cDNA hybrids can be detected by polymerase-based primer extension methods as exemplified herein and known in the art. Alternatively, for array-hybridized mRNA, reverse-transcriptase-based primer extension can be employed. There are several particularly useful advantages of on-array labeling for gene expression analysis. Labeling costs can be dramatically decreased since the amounts of labeled nucleotides employed are substantially less compared to methods for labeling captured targets. Secondly, detection specificity can be increased since a target must both hybridize and also the probe must be extended at its 3′ terminus in a target-specific fashion for label incorporation to occur. Similarly, OLA or primer extension and ligation methods as described further below can be used for detection of hybridized cDNA or mRNA. The latter two methods typically employ the addition of an exogenous nucleic acid for each sequence queried. However, such methods can be useful in applications where the use of primer extension leads to unacceptable levels of ectopic extension.

The above described on-array labeling with primer extension also can be used to monitor alternate splice sites of nucleic acids using the multiplex substrate elements of the invention by, for example, designing the 3′ probe terminus to coincide with a splice junction of a target cDNA or mRNA. The terminus can be placed to uniquely identify all the relevant possible acceptor splice sites for a particular gene. For example, the first 45 bases can be chosen to lie entirely within the donor exon, and the last 5 bases at the 3′ end can lie in a set of possible splice acceptor exons that become spliced adjacent to the first 45 bases. The above exemplary gene expression analysis methods can be found described in, for example, WO 2005/003304 A2, and in U.S. Patent Application Publications 20050181394, 20050059048, 20050053980, 20050037393, 20040259106, 20040259100. Given the teachings and guidance provided herein, these and other expression analysis methods can be beneficially employed in the analysis of gene expression indicative of a pathological condition using the compositions and methods of the invention.

Still further useful methods that can be used in combination with the multiplex substrate elements and methods of the invention include a wide variety of nucleic acid detection, including nucleotide detection methods. As with the above exemplary applications of the invention, any of the analysis or detection methods exemplified herein can be used in combination with any other analyses or with another method well known in the art. Such other methods, or combinations thereof, also can be performed with or without nucleic acid amplification methods. Exemplary nucleic acid detection, nucleotide detection and amplification procedures are described further below.

In a particular nucleic acid detection embodiment, multiplexed, arrayed target specific probes can be modified while hybridized to a probe for detection. Such embodiments include, for example, those utilizing ASPE and SBE as described previously, oligonucleotide ligation assay (OLA), extension ligation, invader technology, or probe cleavage as described in U.S. Pat. No. 6,355,431 B1, U.S. Ser. No. 10/177,727 and/or below. Thus, analyses or detection steps of the invention can be carried out in a mode wherein two or more immobilized target specific probes are modified instead of a target nucleic acid as described previously. Alternatively, detection can include modification of the target nucleic acids while hybridized to their respective target specific probes. Exemplary modifications include those that are catalyzed by an enzyme such as a polymerase.

If desired, an immobilized probe that is not part of a probe-fragment hybrid can be selectively modified compared to a probe-target nucleic acid hybrid. Selective modification of non-hybridized probes can be used to increase assay specificity and sensitivity, for example, by removing probes that are labeled in a template independent manner during the course of a polymerase extension assay. A particularly useful selective modification is degradation or cleavage of single stranded probes that are present in a population or array of probes following contact with target fragments under hybridization conditions. Exemplary enzymes that degrade single stranded nucleic acids include, without limitation, Exonuclease 1 or lambda Exonuclease.

In embodiments utilizing probes with reactive hydroxyls at their 3′ ends and polymerase extension, a useful exonuclease is one that preferentially digests single stranded DNA in the 3′ to 5′ detection. Thus, double stranded probe-target hybrids that form under particular assay conditions are preferentially protected from degradation as is the 3′ overhang of the target that serves as a template for polymerase extension of the probe. However, single stranded probes not hybridized to target under the assay conditions are preferentially degraded. Furthermore, such exonuclease treatment can preferentially degrade single stranded regions of target nucleic acids or other nucleic acids in cases where the fragments or nucleic acids are retained by an array due to interaction with non-probe interacting portions of target nucleic acids. Thus, exonuclease treatment can prevent artifacts that may arise due to a bridged network of 2 or more nucleic acids bound to a probe. Digestion with exonuclease is typically carried out after a probe extension step.

The invention also provides a kit for multiplex nucleic acid detection. The kit includes: (a) a plurality of multiplex substrate elements, each of the multiplex substrate elements including an attached first nucleic acid and an attached second nucleic acid, the first nucleic acid including a first target specific probe and a second nucleic acid including a second target specific probe, and (b) two or more different nucleotides having distinct labels.

The kits of the invention can include some or all of the compositions described or exemplified previously and/or below. Kits of the invention also can include some or all of the compositions, components, reagents and/or preparatory materials used in making or performing a method of the invention. Kits of the invention can additionally include components, reagents, preparatory materials and the like for combining a composition or method of the invention with detection formats or methods other than those exemplified herein, or with other devices or procedures well known in the art. Given the teachings and guidance provided herein, those skilled in the art will understand that kits of the invention can be manufactured to include, for example, a complete repertoire of multiplex substrate elements, probes, labels and reagents for performing one or more nucleic acid detection assays or can include core components such as described above.

Kits of the invention can include a plurality of multiplex substrate elements. Each element can contain, for example, an attached first and second nucleic acid that includes first and second target specific probes as described previously and exemplified in FIG. 1. Similarly, one element within a pair of elements can contain, for example, attached first and second nucleic acids that include first and second target specific probes and a second element can contain, for example, attached third and fourth nucleic acids that include third and fourth target specific probes as described previously and exemplified in FIG. 2. The number of different target specific probes included within the plurality of each kit can include probes specific for particular diagnostic application or include a wide range of different probes generally applicable for detection of alleles or markers for a predetermined percentage of a subject's genome. Therefore, the size of the plurality of multiplex substrate elements can include those ranges and diversity of different probe sequences as exemplified previously.

As with the range of sizes, different number of probe sequences and/or configurations included within a plurality of multiplex substrate elements exemplified above, other components included within a kit of the invention also can include any of the various numbers, sizes, diversities and/or configurations taught or exemplified previously. For example, a kit of the invention can be designed or manufactured for detection of alleles using the configurations exemplified in FIG. 1 or 2. Such detection configurations would employ, for example, two distinct or four distinct labels, respectfully, for detection of the four different nucleotides. Similarly, three or four labels can be included, for example, for detection of triallelic or tetraallelic target nucleic acids as described previously. Therefore, the kits of the invention can include two, three or four different nucleotides having distinct labels with respect to each other.

Multiplex substrate elements included in the kits of the invention can be manufactured with attached first, second, third or fourth target specific probes. Alternatively, the kits can include unattached first, second, third and/or fourth nucleic acids together with a solid support for producing multiplex substrate elements by, for example, chemical coupling or affinity binding. Reagents, instructions or both for coupling or binding the nucleic acids to the solid supports also can be included in such kits of the invention.

Indentifier sequences can be included in the kits of the invention, for use as described previously. Typically, the identifier sequences will be included as part of the first and second target specific probes attached to the multiplex substrate elements. However, those skilled in the art will understand that they can be provided separately and attached via, for example, ligation to some or all of the target specific probes. Alternatively, they can be attached to the multiplex substrate element separate from the first and second target specific probes. As described previously, the identifier sequences for any of the first, second, third or fourth target specific probes can be the same or different with respect to each other.

A kit of the invention also can include any of a number of other components and/or ancillary reagents including, for example, sequencing, detection and/or amplification reagents. A kit can include individual components and/or ancillary reagents or sets of components and/or ancillary reagents. Therefore, the components can be tailored for specific or general applications. Such components and/or ancillary reagents can include, for example, nucleotides including deoxynucleotides and/or dideoxy nucleotides; labels, including sets of two, three or four distinct labels having distinguishable signals; enzymes, including DNA directed polymerase and/or ligase; buffers for sequencing, amplification, washes, storage and the like; labels, probes and nucleic acid standards. In addition to these exemplary components, kits of the invention also can include, for example, substrates for arraying the multiplex substrate elements, slides, tubes, and assay instructions. Therefore, a kit of the invention can include, for example, a plurality of Multiplex substrate elements having attached first, second, third and/or fourth nucleic acids which include target specific probes and a set of distinct probes as well as any combination of components, reagents or preparatory materials for making or using a compositing or method of the invention.

Also provided, is a method of evaluating quality of an array of multiplex substrate elements. The method includes: (a) providing an array including a population of multiplex substrate elements including at least a first and a second subpopulation, wherein the multiplex substrate elements of each subpopulation include: (i) first nucleic acid including a first target specific probe and a first identifier sequence, and (ii) second nucleic acid including a second target specific probe and a second identifier sequence, wherein the first and second nucleic acids are attached to the same multiplex substrate elements; (b) detecting both the first and second identifier sequences to decode the position of each of the target specific probes on the array, and (c) determining whether the amount of each hybridizable target specific probe at each multiplex substrate element is sufficient to pass a quality metric, wherein the amount of each said first and second identifier sequence at each multiplex substrate element correlates with the amount of each target specific probe available for hybridization at each multiplex substrate element.

The compositions and methods of the invention can be usefully employed in quality control of arrays preparations and array manufacturing processes. The identifier sequences attached to a population of multiplex substrate elements can be generated to contain two or more different subpopulations as described previously. Each subpopulation can be detected by decoding to determine whether the amount the identifier correlates with the amount of its corresponding target specific probe. The greater the correlation between first, second, third and/or fourth identifier sequence with first, second, third and/or fourth target specific probe, respectively indicates higher quality in multiplex substrate element production and greater uniformity across different element types.

Standards for assessing whether the amount of each target specific probe at each multiplex substrate element is sufficient to pass a quality metric are well know in the art. Quality metrics can include thresholds for individual target specific probes, thresholds for probe amounts constituting a subpopulation of multiplex substrate elements, thresholds for probe amounts for a population of multiplex substrate elements or any combination, including all of the above criteria or any combination thereof. Useful quality metrics applicable to the method of the invention for evaluating array quality include, for example, the presence of expected identifier sequences, threshold for a minimum expected signal for decoder binding ligands that are complementary to identifier sequences or ratio of signals for one decoder binding ligand to a second decoder binding ligand where two decoder binding ligands bind to different identifier sequences on the same multiplex substrate element. In a particular embodiment array quality can be evaluated by calculating whether an identifier binding ligand when hybridized to a defined concentration of labeled decoder binding ligand generates signal exceeding a threshold and if the ratio of such signals from two segments of the array is equal to a value of one plus or minus a defined interval.

Detecting and determining the amount of target specific probes attached to multiplex substrate elements can be performed as described above. Detection and determination of the amount of associated identifier sequence can be performed by any method for nucleic acid detection well known in the art including, for example, those exemplified previously. Decoding the identifier sequence within each subpopulation can be a particularly useful detection step for evaluating the quality of an array because this method also can be employed for identifying the location of a multiplex substrate element within the plurality of arrayed elements.

Decoding populations, including complex populations, of nucleic acid sequences is well known in the art and can be found described in, for example, U.S. Pat. No. 7,033,754; or US 2003/0157504 and Gunderson et al., Genome Research 14: 870-77 (2004), each of which is incorporated herein by reference. Any of such well known methods for decoding can be equally employed in a method of evaluating the quality of an array or as a method of identifying a multiplex substrate element. Briefly, decoding nucleic acids can be employed to detect identifier sequences by nucleic acid hybridization methods well know in the art and exemplified previously. The decoder nucleic acids are synthesized to be complementary to their cognate identifier sequence so as to specifically hybridize. Detection of the decoder sequence will indicate the presence and/or amount of its complementary identifier sequence and its corresponding target specific probe. In like fashion, complementary decoder sequences can be produced for each identifier sequence within a multiplex substrate element subpopulation for detection and correlation of the amount of identifier sequence with the amount of associated target specific probe. Similarly, in decoding applications, complementary decoder sequences can be used to detect and determine the presence and/or location of one or more multiplex substrate elements within a subpopulation or within all subpopulations of the array.

The invention further provides a method for identifying a plurality of target nucleic acid sequences. The method includes: (a) obtaining signals from a plurality of multiplex substrate elements, each of the multiplex substrate elements comprising two different target specific probes, the signals comprising a first signal indicative of a first type of nucleotide in a first target nucleic acid and a second signal indicative of a second type of nucleotide in a second target nucleic acid, wherein the signals are distinguishable from each other, and wherein the first type of nucleotide is different from the second type of nucleotide; (b) providing nucleotide sequences for the two different target specific probes at each of the multiplex substrate elements; (c) determining the presence or absence of the first signal and the second signal at each of the multiplex substrate elements, wherein at least a subset of the multiplex substrate elements produce the first signal and the second signal, thereby determining the type of nucleotide at each of the multiplex substrate elements, and (d) correlating the nucleotide sequences for the two different target specific probes with the type of nucleotide at each of the multiplex substrate elements, thereby identifying the nucleotide sequences of the first target nucleic acid sequence and the second target nucleic target sequence at each of the multiplex elements.

Methods for detecting and delineating signals from different target specific probes having distinct labels within a mixture of multiplex substrate elements are similar to those described above for decoding an identifier sequence and can be equally employed for detection of both simple and complex mixtures of discrete labels incorporated into modified target specific probes of the invention. For example, in a decoding format, the signal is derived from a complementary decoder sequence specifically hybridized to its corresponding identifier sequence where different decoders can employ different labels. In a target detection format employing, for example, the genotyping methods exemplified previously, the signal is derived from label incorporation into a target specific probe through enzymatic incorporation during performance of ASPE, ASSBE, SBE and similar methods of nucleotide and/or nucleic acid detection. Therefore, signal detection devices, filters, computational algorithms, computational resources and associated automation for decoding identifier sequences also can be equally employed for signal detection arising from the methods of detecting nucleic acids of the invention employing multiplex substrate elements having, for example, first, second, third and/or fourth target specific probes and utilizing, for example, at least two, three, or four distinct labels as described previously and exemplified in FIGS. 1 and 2.

Briefly, for example, a plurality of multiplex substrate elements can be employed in a nucleic acid detection method as exemplified previously. Following the illustration of FIG. 1, for example, labels can be used that are indicative of an incorporated nucleotide in a modified target specific probe. Therefore, following the methods of the invention, determining the presence or absence of incorporated label can be used to determine both the presence or absence of a first or second target nucleic acid sequence as well as to identify the nucleotide sequence of first and second target nucleic acid sequences. For example, a first signal arising from label incorporation into first target specific probe of a multiplex substrate element within a plurality will be indicative of a first type of nucleotide. A second signal arising from label incorporation into the second target specific probe of the multiplex substrate element will be indicative of a second, different type of nucleotide. Determination of nucleotide sequences for the target nucleic acids requires correlation of the signal through the modified target specific probe to the target nucleic acid as described previously.

For example, the methods for identifying a plurality of target nucleic acid sequences of the invention include obtaining signals from a plurality of multiplex substrate elements as described above. By way of exemplification using a label management scheme where each label is unique to a specific nucleotide, each signal will be indicative of a single nucleotide type. Thus, a first signal will indicate that a first type of nucleotide was added to a first target specific probe in the presence of a first target nucleic acid. Similarly, a second, third or fourth distinguishable signal will indicate that a second, third or fourth type of nucleotide was added to a target specific probe, respectively. Determination of the signal type and its presence or absence therefore determines the type of nucleotide incorporated into first and second target specific probes in the presence of first and second target nucleic acids, for example. By correlation, the signal also is determinative of the incorporated nucleotide and complementary to the corresponding nucleotide in the target nucleic acid.

By way of exemplification using a label management scheme where each label is indicative of two types of nucleotides, a first and second multiplex substrate element having first, second, third and fourth target specific probes can be employed to determine the presence or absence of a nucleotide incorporated into the target specific probes as described previously. By correlation, the resultant signal also is indicative of the corresponding nucleotide in the target nucleic acid.

Briefly, first and third target specific probes, for example, hybridize to different alleles (ie, first and second) of the same locus (ie, a first locus) and second and fourth target specific probes, for example, hybridize to different alleles (ie, first and second) of the same locus, but which is different than the first locus (ie, a second locus). In this embodiment, the sequence of the first allele is identified by distinguishing presence or absence of the first signal at the first and second multiplex element and the sequence of the second allele is identified by distinguishing presence or absence of the second signal at the first and second multiplex element.

Detection, determination of signals and correlations procedures exemplified above and described previously can be performed on some or all of the multiplex substrate elements within a plurality to identify nucleotide sequences for some or all target nucleic acids within a sample mixture. Using computational systems such as those previously exemplified for signal detection, identifications can be made in parallel, series or simultaneously for rapid and efficient multiplex determination of a multitude of different target nucleic acids. Automation using devices and systems well known in the art such as robotics and related computational algorithms and executable code also can be employed to further increase the speed, efficiency and throughput of a large plurality of target nucleic acids for sequence determination. Accordingly, algorithms and executable code for data retrieval processing and integration can be used in conjunction with the systems and methods described herein for obtaining signals, providing nucleotide sequences for some or all modified target specific probes, determining the presence or absence of signals arising from some or all multiplex substrate elements and correlating nucleotide sequences for identifying target nucleic acid sequences.

Throughout this application various publications have been referenced within parentheses. The disclosures of these publications in their entireties are hereby incorporated by reference in this application in order to more fully describe the state of the art to which this invention pertains.

It is understood that modifications which do not substantially affect the activity of the various embodiments of this invention are also included within the definition of the invention provided herein. Those skilled in the art will readily appreciate that the specific examples and studies detailed above are only illustrative of the invention. Accordingly, specific examples disclosed herein are intended to illustrate but not limit the present invention. It also should be understood that, although the invention has been described with reference to the disclosed embodiments, various modifications can be made without departing from the spirit of the invention. Accordingly, the invention is limited only by the following claims.

1-54. (canceled)

55. A method for independently detecting the alleles of at least two separate polymorphisms on each bead of a plurality of different beads, comprising:
(a) providing a plurality of beads distributed on a substrate, wherein each bead has a different predetermined set comprising
(1) a first nucleic acid, comprising a first target-specific portion corresponding to a first polymorphism of either a first or a second allele,
(2) a second nucleic acid, comprising a second target-specific portion corresponding to a second polymorphism of either a third or a fourth allele wherein each of the four alleles are different nucleotides;
(b) contacting the plurality of beads with target nucleic acids having first and second target portions, wherein
(1) a first target portion hybridizes to the first target-specific portion of a bead,
(2) a second target portion hybridizes to the second target-specific portion of the same bead;
(c) contacting the hybridized target portions with a mixture of distinguishably labeled first, second, third and fourth nucleotides and a template-directed enzyme that incorporates for each of the beads:
(1) the first labeled nucleotide to the first target-specific portion if the first polymorphism is the first allele,
(2) the second labeled nucleotide to the first target-specific portion if the first polymorphism is the second allele,
(3) the third labeled nucleotide to the second target-specific portion if the second polymorphism is the third allele, and
(4) the fourth labeled nucleotide to the second target-specific portion if the second polymorphism is the fourth allele; and
(d) independently detecting incorporated labeled nucleotides on each bead, thereby independently detecting the alleles of at least two separate polymorphisms on each bead of a plurality of beads.

56. The method of claim 55, wherein the beads are randomly distributed.

57. The method of claim 56, wherein each of the beads is labeled with a detectable label.

58. The method of claim 57, wherein the detectable label is a holographic pattern.

59. The method of claim 57, wherein the detectable label is a fluorophore.

60. The method of claim 57, wherein the detectable label is a quantum dot.

61. The method of claim 56, wherein each bead further comprises an identifier sequence.

62. The method of claim 61, wherein one nucleic acid of a bead comprises the identifier sequence.

63. The method of claim 62, wherein the other nucleic acid of the bead comprises a second identifier sequence.

64. The method of claim 56, further comprising identifying the location of the bead.

65. The method of claim 55, wherein the template-directed enzyme is a polymerase.

66. The method of claim 65, wherein detecting step (d) comprises an allele-specific polymerase extension assay (ASPE).

67. The method of claim 65, wherein detecting step (d) comprises a single-base extension assay (SBE).

68. The method of claim 55, wherein the labeled nucleotides are part of oligonucleotides.

69. The method of claim 68, wherein the template-directed enzyme is a ligase

70. The method of claim 69, wherein detecting step (d) comprises an oligonucleotide ligation assay (OLA).
